(19) United States
(12) Patent Application Publication
(10) Pub. No.: US 2018/0263594 A1
(43) Pub. Date: Sep. 20, 2018

(54) SEMICONDUCTOR DEVICE, ULTRASONIC IMAGE PICKUP DEVICE, SEMICONDUCTOR DEVICE MANUFACTURING METHOD, AND ULTRASONIC IMAGING SYSTEM

(71) Applicant: SONY CORPORATION, TOKYO (JP)

(72) Inventor: RUI MORIMOTO, KANAGAWA (JP)

(21) Appl. No.: 15/760,068

(22) PCT Filed: Jul. 15, 2016
(86) PCT No.: PCT/JP2016/003366

§ 371 Date: Mar. 14, 2018

(30) Foreign Application Priority Data
Sep. 29, 2015 (JP) 2015-190920

(51) Int. Cl. A61B 8/14; H01L 21/762; H01L 21/8234; H01L 27/06; H01L 27/088; H01L 27/12; H01L 29/786; H01L 29/868

(52) U.S. Cl. CPC A61B 8/14; H01L 21/762; H01L 21/8234; H01L 29/868; H01L 27/088; H01L 27/12; H01L 29/786; H01L 27/06

(57) ABSTRACT

[Object] To provide a semiconductor device including a protection circuit that exhibits satisfactory performance even in a small area, an ultrasonic image pickup device, a semiconductor device manufacturing method, and and an ultrasonic imaging system. [Solving Means] The semiconductor device according to the present technology includes an integrated circuit formed on an SOI substrate including a silicon substrate formed of crystalline silicon, a BOX layer laminated on the silicon substrate, and an SOI layer laminated on the BOX layer, the semiconductor device including: a protection circuit; and an element separation region. The protection circuit constitutes the integrated circuit and includes a semiconductor region having the same crystal orientation as the silicon substrate. The element separation region penetrates the SOI substrate and separates the protection circuit.

### TECHNICAL FIELD

The present technology relates to a semiconductor device including an integrated circuit including protection circuits, an ultrasonic image pickup device, a semiconductor device manufacturing method, and an ultrasonic imaging system.

### BACKGROUND ART

Ultrasonic imaging refers to generation of ultrasonic images by applying ultrasonic waves from an ultrasonic transducer to a measurement target, and detecting reflected waves generated from the measurement target with the ultrasonic transducer. The ultrasonic imaging has been utilized in ultrasonic endoscopes and ultrasonic catheters.

In this context, there is a significant difference between drive voltage to be applied to the ultrasonic transducer such that the ultrasonic waves are generated, and signal voltage to be generated by the ultrasonic transducer through the detection of the ultrasonic waves. For example, the drive voltage is at most approximately several hundred V, and the signal voltage is at approximately several μV.

In view of such circumstances, an amplifier circuit for amplifying the signal voltage is utilized. Meanwhile, when the drive voltage is applied to the amplifier circuit, the amplifier circuit fails. As a countermeasure, protection circuits that prevent the drive voltage from reaching the amplifier circuit are also needed. When these circuits are implemented to a single semiconductor circuit, an implementation space can be saved.

In this context, an SOI (silicon on insulator) substrate, which is excellent in voltage resistance, is suited to use as the semiconductor substrate including both the amplifier circuit and the protection circuits. For example, Patent Literature 1 discloses a semiconductor device obtained by implementing the amplifier circuit and the protection circuits to the single SOI substrate. In this semiconductor device, the amplifier circuit is formed on the SOI substrate, and the protection circuits are formed in through-holes formed in the SOI substrate. The protection circuits are made of polysilicon filled in the through-holes.

### CITATION LIST

### Patent Literature

Patent Literature 1: Japanese Patent Application Laid-open No. 2010-50156

### DISCLOSURE OF INVENTION

### Technical Problem

However, there is a disadvantage that polysilicon generally has high resistance, and hence diodes having satisfactory leakage-current characteristics are difficult to form owing to defects in polysilicon. Further, the SOI substrate generally has a thickness of approximately 0.8 mm, and a manufacturing process of forming the through-holes each having a width of several ten μm in such an SOI substrate is difficult to execute. When the widths of the through-holes are increased to relax an aspect ratio (opening/depth), areas of elements are difficult to reduce.

In view of such circumstances, the present technology has been made to achieve an object to provide a semiconductor device including a protection circuit that exhibits satisfactory performance even in a small area, an ultrasonic image pickup device, a semiconductor device manufacturing method, and and an ultrasonic imaging system.

### Solution to Problem

In order to achieve the above-mentioned object, according to an embodiment of the present technology, there is provided a semiconductor device including an integrated circuit formed on an SOI substrate including
a silicon substrate formed of crystalline silicon,
a BOX (buried oxide) layer laminated on the silicon substrate, and
an SOI (silicon on insulator) layer laminated on the BOX layer, the semiconductor device including:
a protection circuit; and
an element separation region.

The protection circuit constitutes the integrated circuit and includes a semiconductor region having the same crystal orientation as the silicon substrate.

The element separation region penetrates the SOI substrate and separates the protection circuit.

With this configuration, a semiconductor device in which an integrated circuit including a protection circuit is formed on a single SOI substrate can be provided. The semiconductor region of this protection circuit is made of the crystalline silicon having the same crystal orientation as the silicon substrate. The crystalline silicon is higher in movability than amorphous silicon such as polysilicon. Thus, it is possible to reduce areas of elements of the protection circuit, and to secure satisfactory leakage-current characteristics.

The protection circuit may be a diode.

This configuration enables the diode to serve as a TR (transmit-receive) switch, and the diode can be utilized as the protection circuit.

The protection circuit may be a vertical transistor.

This configuration enables the vertical transistor to serve as the TR switch, and the vertical transistor can be utilized as the protection circuit.

The element separation region may be formed of any one or two or more of silicon oxide, silicon nitride, and polysilicon.

The element separation region can be formed by forming a silicon oxide film or a silicon nitride film in a through-hole formed in the SOI substrate, and by filling polysilicon therein.

The element separation region may include a gate electrode of the vertical transistor.

By connecting wires to the polysilicon filled in the element separation region, this polysilicon can be utilized as the gate electrode of the vertical transistor.

The SOI substrate may include a first surface and a second surface on a side opposite to the first surface,
the protection circuit may include a first semiconductor element and a second semiconductor element,
the first semiconductor element may be formed by laminating a first semiconductor region that is on the first surface side and is of a first impurity type, and a second semiconductor region that is on the second surface side and is of a second impurity type, and
the second semiconductor element may be formed by laminating a third semiconductor region that is on the first surface side and is of the second impurity type, and a fourth semiconductor region that is on the second surface side and is of the first impurity type.

This configuration enables the first semiconductor element and the second semiconductor element to serve as a back-to-back diode. The back-to-back diode refers to a pair of diodes configured in a manner that a P-type semiconductor region of one is connected to an N-type semiconductor region of another. In a large number of high-voltage elements, this back-to-back diode is used as an element having a function of a Zener diode.

The above-described semiconductor device may further include
a ground contact structure that is provided on the first surface of the semiconductor device and is electrically conducted to the first semiconductor region and the third semiconductor region.

With this, wire routing to the back-to-back diode is simplified. As a result, improvement in yield, reduction in manufacturing cost, and enhancement in wiring reliability can be achieved.

The ground contact structure may include a ground wire that is connected to the first semiconductor region and the third semiconductor region, and is common to both the first semiconductor region and the third semiconductor region.

Potentials of adjacent ones of the semiconductor regions (first semiconductor region and third semiconductor region) of the back-to-back diode are equal to each other. Thus, both the regions can be connected to each other with the common ground wire.

The ground contact structure may include a ground electrode that is connected to the ground wire, and is common to both the first semiconductor region and the third semiconductor region.

This configuration enables the first semiconductor element and the second semiconductor element to be conducted to the common ground electrode.

The above-described semiconductor device may further include
a signal wire that is connected to the second semiconductor region and the fourth semiconductor region, and is common to both the second semiconductor region and the fourth semiconductor region.

Potentials of other adjacent ones of the semiconductor regions (second semiconductor region and foruth semiconductor region) of the back-to-back diode are equal to each other. Thus, both the regions can be connected to each other with the common signal wire

In order to achieve the above-mentioned object, according to another embodiment of the present technology, there is provided an ultrasonic image pickup device including
a semiconductor device.

This semiconductor device includes an integrated circuit formed on an SOI substrate including
a silicon substrate formed of crystalline silicon,
a BOX layer laminated on the silicon substrate, and
an SOI layer laminated on the BOX layer, the semiconductor device including
   a protection circuit that configures the integrated circuit and includes a semiconductor region having the same crystal orientation as the silicon substrate, and
   an element separation region that penetrates the SOI substrate and separates the protection circuit.

This semiconductor device can be utilized as an impedance matching circuit of an ultrasonic transducer of the ultrasonic image pickup device.

According to still another embodiment of the present technology, there is provided a manufacturing method for a semiconductor device including an integrated circuit formed on an SOI substrate, the manufacturing method including:
preparing the SOI substrate including
   a silicon substrate formed of crystalline silicon,
   a BOX layer laminated on the silicon substrate, and
   an SOI layer laminated on the BOX layer;
forming, by an epitaxial crystal growth method, a protection circuit that configures the integrated circuit and includes a semiconductor region that has the same crystal orientation as the silicon substrate, on the silicon substrate; and
forming an element separation region that penetrates the SOI substrate and separates the protection circuit.

In the forming of the protection circuit, there may be used a substrate polishing method of polishing the silicon substrate from a surface on a side opposite to a surface on a side where crystal growth of the semiconductor region progresses, to expose the semiconductor region.

According to yet another embodiment of the present technology, there is provided an ultrasonic imaging system including
an ultrasonic catheter.

This ultrasonic catheter includes a semiconductor device including an integrated circuit formed on an SOI substrate including
a silicon substrate formed of crystalline silicon,
a BOX layer laminated on the silicon substrate, and
an SOI layer laminated on the BOX layer, the semiconductor device including
a protection circuit that configures the integrated circuit and includes a semiconductor region having the same crystal orientation as the silicon substrate, and
an element separation region that penetrates the SOI substrate and separates the protection circuit.

According to yet another embodiment of the present technology, there is provided an ultrasonic imaging system including:
an intraoperative ultrasonic probe; or
an ultrasound endoscope.

This intraoperative ultrasonic probe or this ultrasound endoscope includes a semiconductor device including an integrated circuit formed on an SOI substrate including
a silicon substrate formed of crystalline silicon,
a BOX layer laminated on the silicon substrate, and
an SOI layer laminated on the BOX layer, the semiconductor device including
a protection circuit that configures the integrated circuit and includes a semiconductor region having the same crystal orientation as the silicon substrate, and
an element separation region that penetrates the SOI substrate and separates the protection circuit.

According to yet another embodiment of the present technology, there is provided an ultrasonic imaging system including
a hand-held instrument that has an ultrasonic imaging function and is used in laparoscopic surgery.

This hand-held instrument having the ultrasonic imaging function includes a semiconductor device including an integrated circuit formed on an SOI substrate including
a silicon substrate formed of crystalline silicon,
a BOX layer laminated on the silicon substrate, and
an SOI layer laminated on the BOX layer, the semiconductor device including
a protection circuit that configures the integrated circuit and includes a semiconductor region having the same crystal orientation as the silicon substrate, and
an element separation region that penetrates the SOI substrate and separates the protection circuit.

According to yet another embodiment of the present technology, there is provided an ultrasonic imaging system including
robotic forceps that have an ultrasonic imaging function and are used in laparoscopic surgery

These robotic forceps having the ultrasonic imaging function include a semiconductor device including an integrated circuit formed on an SOI substrate including
a silicon substrate formed of crystalline silicon,
a BOX layer laminated on the silicon substrate, and
an SOI layer laminated on the BOX layer, the semiconductor device including
a protection circuit that configures the integrated circuit and includes a semiconductor region having the same crystal orientation as the silicon substrate, and
an element separation region that penetrates the SOI substrate and separates the protection circuit.

### Advantageous Effects of Invention

As described hereinabove, according to the present technology, a semiconductor device including a protection circuit that exhibits satisfactory performance even in a small area, an ultrasonic image pickup device, a semiconductor device manufacturing method, and an ultrasonic imaging system can be provided. Note that, the advantages disclosed herein are not necessarily limited to those described hereinabove, and all of the advantages disclosed herein can be obtained.

### BRIEF DESCRIPTION OF DRAWINGS

FIG. 1 A cross-sectional view of a semiconductor device according to a first embodiment of the present technology.

FIG. 2 A cross-sectional view of a part of a configuration of the semiconductor device.

FIG. 3 A cross-sectional view of an SOI substrate that is used for preparing the semiconductor device.

FIG. 4 A plan view of an element separation region of the semiconductor device.

FIG. 5 A schematic diagram of an impedance matching circuit that utilizes the semiconductor device.

FIG. 6 A schematic view illustrating a manufacturing method for the semiconductor device.

FIG. 7 Another schematic view illustrating the manufacturing method for the semiconductor device.

FIG. 8 Still another schematic view illustrating the manufacturing method for the semiconductor device.

FIG. 9 Yet another schematic view illustrating the manufacturing method for the semiconductor device.

FIG. 10 Yet another schematic view illustrating the manufacturing method for the semiconductor device.

FIG. 11 A schematic view illustrating another manufacturing method for the semiconductor device.

FIG. 12 Another schematic view illustrating the other manufacturing method for the semiconductor device.

FIG. 13 A cross-sectional view of a semiconductor device according to a first modification of the first embodiment.

FIG. 14 A cross-sectional view of a semiconductor device according to a second modification of the first embodiment.

FIG. 15 A schematic view of an IVUS (intravascular ultrasound endoscope) that utilizes the semiconductor devices according to the present technology.

FIG. 16 A schematic view of an IVUS having a general structure.

FIG. 17 A cross-sectional view of a semiconductor device according to a second embodiment of the present technology.

FIG. 18 A cross-sectional view of a part of a configuration of the semiconductor device.

FIG. 19 A schematic diagram of an impedance matching circuit that utilizes the semiconductor device.

FIG. 20 A schematic view of an intraoperative ultrasonic probe that utilizes the semiconductor devices according to the present technology.

FIG. 21 A schematic view of an intraoperative ultrasonic probe having a general structure.

FIG. 22 A schematic view of a laparoscopic surgical holder that utilizes the semiconductor devices according to the present technology.

FIG. 23 A schematic view of a laparoscopic surgical holder having a general structure.

FIG. 24 A schematic view of a handle portion of a laparoscopic surgical holder that utilizes the semiconductor devices according to the present technology.

FIG. 25 A schematic view of robotic forceps of a laparoscopic-surgery surgical robot that utilizes the semiconductor devices according to the technology.

FIG. 26 A schematic view of robotic forceps of a laparoscopic-surgery surgical robot having a general structure.

### MODES FOR CARRYING OUT THE INVENTION

### First Embodiment

A semiconductor device according to a first embodiment of the present technology is described.

### Configuration of Semiconductor Device

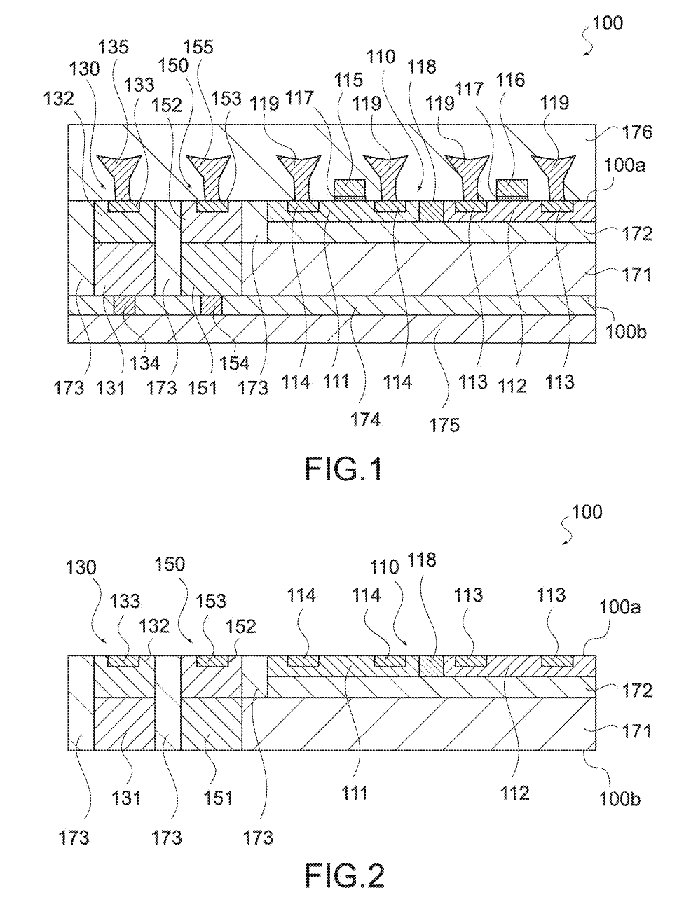

FIG. 1 is a cross-sectional view of a configuration of a semiconductor device 100 according to this embodiment, and FIG. 2 is a cross-sectional view of a part of the configuration of the semiconductor device 100. As illustrated in these drawings, the semiconductor device 100 includes an LV (Low Voltage) circuit 110, a first diode 130, a second diode 150, a silicon substrate 171, a BOX (buried oxide: buried oxide film) layer 172, an element separation region 173, a bottom-surface insulating layer 174, a ground electrode 175, and a top-surface insulating layer 176.

The semiconductor device 100 is an integrated circuit including the LV circuit 110, the first diode 130, and the second diode 150. The first diode 130 and the second diode 150 serve as protection circuits of this integrated circuit.

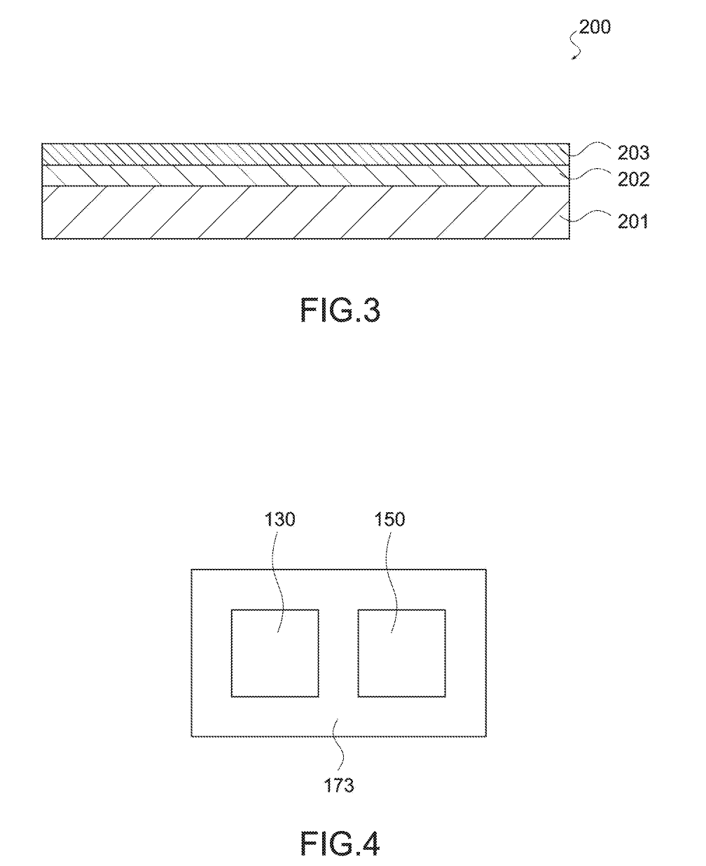

The semiconductor device 100 can be prepared from a single SOI substrate. FIG. 3 is a schematic view of an SOI substrate 200 of the semiconductor device 100. As illustrated in FIG. 3, the SOI substrate 200 includes a silicon substrate 201, a BOX layer 202, and an SOI layer 203.

The silicon substrate 201 is made of P-type crystalline silicon. The BOX layer 202, which is made of SiO₂, is laminated on the BOX layer 202. The SOI layer 203, which is made of silicon, is laminated on the BOX layer 202. The SOI substrate 200 can be prepared by a SIMOX (Separation by IMplantation of OXygen) method or by a bonding method.

The semiconductor device 100 is prepared by executing a processing process described below on the SOI substrate 200. The silicon substrate 171 of the semiconductor device 100 corresponds to a part of the silicon substrate 201 of the SOI substrate 200, and the BOX layer 172 of the semiconductor device 100 corresponds to a part of the BOX layer 202 of the SOI substrate 200.

In the following, among parts that are formed by processing the SOI substrate 200 of the semiconductor device 100 (structure of FIG. 2), a surface on the LV circuit 110 side is referred to as a top surface 100a, and a surface on a side opposite thereto is referred to as a bottom surface 100b.

The LV circuit 110 includes an N-type semiconductor region 111, a P-type semiconductor region 112, N⁺⁺-type semiconductor regions 113, P⁺⁺-type semiconductor regions 114, a first gate electrode 115, a second gate electrode 116, gate insulating films 117, an element separation layer 118, and signal wires 119.

The N-type semiconductor region 111, which is made of silicon doped with an N-type dopant, is laminated on the BOX layer 172. A typical N-type dopant is phosphorus. The P-type semiconductor region 112, which is made of silicon doped with a P-type dopant, is laminated on the BOX layer 172. A typical P-type dopant is boron. The N-type semiconductor region 111 and the P-type semiconductor region 112 are exposed on the top surface 100a, and are separated from each other by the element separation layer 118 made of SiO₂.

The N⁺⁺-type semiconductor regions 113 are made of silicon doped with a large amount of the N-type dopant, and are formed apart from each other at two positions in the P-type semiconductor region 112. The N⁺⁺-type semiconductor regions 113 are exposed on the top surface 100a, and are connected to the signal wires 119.

The P⁺⁺-type semiconductor regions 114 are made of silicon doped with a large amount of the P-type dopant, and are formed apart from each other at two positions in the N-type semiconductor region 111. The P⁺⁺-type semiconductor regions 114 are exposed on the top surface 100a, and are connected to the signal wires 119.

The first gate electrode 115 is made of metals such as aluminum or conductive materials such as polysilicon, and is formed on the N-type semiconductor region 111 through intermediation of the gate insulating film 117. The second gate electrode 116 is made of the metals such as aluminum or the conductive materials such as polysilicon, and is formed on the P-type semiconductor region 112 through intermediation of the gate insulating film 117.

The N-type semiconductor region 111, the P-type semiconductor region 112, the N⁺⁺-type semiconductor regions 113, and the P⁺⁺-type semiconductor regions 114 are regions formed by implanting the P-type dopant or the N-type dopant into the SOI layer 203 of the SOI substrate 200. The element separation layer 118 is a region formed by oxidizing the SOI layer 203 into SiO₂.

Note that, the configuration of the LV circuit 110 is not limited to the above-described configuration as long as the configuration of the LV circuit 110 can be prepared by processing the SOI layer 203 of the SOI substrate 200.

The first diode 130 includes an N-type semiconductor region 131, a P-type semiconductor region 132, a P⁺⁺-type semiconductor region 133, a ground wire 134, and a signal wire 135. The N-type semiconductor region 131 is made of the silicon doped with the N-type dopant, and the P-type semiconductor region 132 is made of the silicon doped with the P-type dopant. The P⁺⁺-type semiconductor region 133 is made of the silicon doped with the large amount of the P-type dopant. In other words, the N-type semiconductor region 131 is a semiconductor region of a first impurity type (N type), and the P-type semiconductor region 132 and the P⁺⁺-type semiconductor region 133 are each a semiconductor region of a second impurity type (P type).

The N-type semiconductor region 131 and the P-type semiconductor region 132 are laminated on each other. The N-type semiconductor region 131 is exposed on the bottom surface 100b, and the P-type semiconductor region 132 is exposed on the top surface 100a. The P⁺⁺-type semiconductor region 133 is formed in the P-type semiconductor region 132, and is exposed on the top surface 100a.

The N-type semiconductor region 131, the P-type semiconductor region 132, and the P⁺⁺-type semiconductor region 133 are each made of the crystalline silicon, and have the same crystal orientation as the silicon substrate 171. This is because these semiconductor regions are each made of the crystalline silicon that is formed by the implantation of the dopants into parts of the silicon substrate 201, or formed by an epitaxial crystal growth method on the silicon substrate 201.

On the bottom surface 100b, the ground wire 134 is connected to the N-type semiconductor region 131. On the top surface 100a, the signal wire 135 is connected to the P⁺⁺-type semiconductor region 133.

The second diode 150 includes a P-type semiconductor region 151, an N-type semiconductor region 152, an N⁺⁺-type semiconductor region 153, a ground wire 154, and a signal wire 155. The P-type semiconductor region 151 is made of the silicon doped with the P-type dopant, and the N-type semiconductor region 152 is made of the silicon doped with the N-type dopant. The N⁺⁺-type semiconductor region 153 is made of the silicon doped with the large amount of the N-type dopant. In other words, the P-type semiconductor region 151 is the semiconductor region of the second impurity type (P type), and the N-type semiconductor region 152 and the N⁺⁺-type semiconductor region 153 are each the semiconductor region of the first impurity type (N type).

The P-type semiconductor region 151 and the N-type semiconductor region 152 are laminated on each other. The P-type semiconductor region 151 is exposed on the bottom surface 100b, and the N-type semiconductor region 152 is exposed on the top surface 100a. The N⁺⁺-type semiconductor region 153 is formed in the N-type semiconductor region 152, and is exposed on the top surface 100a.

The P-type semiconductor region 151, the N-type semiconductor region 152, and the N⁺⁺-type semiconductor region 153 are each made of the crystalline silicon, and have the same crystal orientation as the silicon substrate 171. This is because these semiconductor regions are each made of the monocrystalline silicon that is formed by the implantation of the dopants into parts of the silicon substrate 201, or formed by the epitaxial crystal growth method on the silicon substrate 201.

On the bottom surface 100b, the ground wire 154 is connected to the P-type semiconductor region 151. On the top surface 100a, the signal wire 155 is connected to the N⁺⁺-type semiconductor region 153.

The element separation region 173 separates the first diode 130 and the second diode 150 from each other. The element separation region 173 penetrates from the top surface 100a to the bottom surface 100b. FIG. 4 is a schematic view of the element separation region 173 as viewed from the top surface 100a side. As illustrated in FIG. 4, the element separation region 173 is formed around the first diode 130 and the second diode 150.

The element separation region 173 is made of a material of any one or two or more of silicon oxide, silicon nitride, or polysilicon. For example, the element separation region 173 may have a structure obtained by forming a film of insulating materials such as silicon oxide or silicon nitride in a through-hole formed in the SOI substrate 200, and by filling the hole with polysilicon.

The bottom-surface insulating layer 174 is arranged on the bottom surface 100b so as to prevent diffusion of moisture or impurity. The bottom-surface insulating layer 174 is made of, for example, p-SiO (silicon oxide formed by plasma-enhanced chemical vapor deposition). The bottom-surface insulating layer 174 is patterned such that the N-type semiconductor region 131 and the P-type semiconductor region 151 are exposed, and that the bottom-surface insulating layer 174 has opening portions in which the ground wire 134 and the ground wire 154 are formed.

The ground electrode 175 is arranged on the bottom-surface insulating layer 174, and is connected to the ground wire 134 and the ground wire 154. With this, the ground electrode 175 is electrically conducted to the N-type semiconductor region 131 and the P-type semiconductor region 151. In this way, the ground electrode 175 forms, cooperatively with the ground wire 134 and the ground wire 154, a ground contact structure for the first diode 130 and the second diode 150. The ground electrode 175 is made of the conductive materials such as aluminum.

The top-surface insulating layer 176 is arranged on the top surface 100a so as to seal the circuits. The top-surface insulating layer 176 is made of the insulating materials such as SiO₂.

### Utilization Example of Semiconductor Device

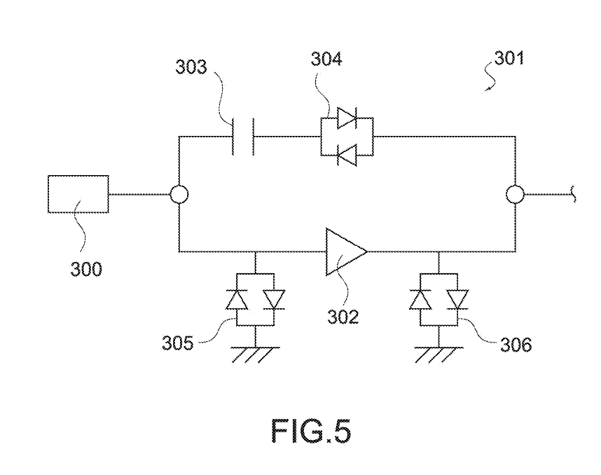

FIG. 5 is a schematic diagram of a circuit configuration of an impedance matching circuit 301 of an ultrasonic transducer 300 that is capable of utilizing the semiconductor device 100.

As shown in FIG. 5, the impedance matching circuit 301 includes an amplifier 302, a capacitor 303, a first TR (transmit-receive) switch 304, a second TR switch 305, and a third TR switch 306. The first TR switch 304, the second TR switch 305, and the third TR switch 306 are each a back-to-back diode. The back-to-back diode refers to a pair of diodes arranged, as shown in FIG. 5, in a manner that a P-type semiconductor region of one is connected to an N-type semiconductor region of another.

A drive signal to the ultrasonic transducer 300 reaches the ultrasonic transducer 300 via the first TR switch 304 and the capacitor 303, and causes the ultrasonic transducer 300 to generate ultrasonic waves. The second TR switch 305 and the third TR switch 306 prevent the drive signal from reaching the amplifier 302. Note that, depending on how the transducer needs to be driven, the capacitor 303 may be short-circuited.

When reflected waves of the ultrasonic waves reach the ultrasonic transducer 300, the ultrasonic transducer 300 generates a detection signal. The detection signal is amplified by the amplifier 302, and then output.

In this way, the impedance matching circuit 301 includes the amplifier 302 being an amplifier circuit, the first TR switch 304, the second TR switch 305, and the third TR switch 306 being protection circuits.

In the semiconductor device 100, the LV circuit 110 can be utilized as the amplifier 302, and the first diode 130 and the second diode 150 can be utilized as the TR switch. With this, the impedance matching circuit 301 can be provided in the single semiconductor device 100.

Note that, the first diode 130 and the second diode 150 constitute one of the three TR switches. Similar to the first diode 130 and the second diode 150, other two of the switches can also be formed in the semiconductor device 100.

Further, as described above, the first diode 130 and the second diode 150 constitute the back-to-back diode. In a large number of high-voltage elements, the back-to-back diode is used as an element having a function of a Zener diode. When the diodes are unidirectional, wires need to be connected to the diodes from a side surface of a substrate, or through-wires need to be formed and connected thereto. Thus, there are a risk of a decrease in yield and an increase in cost in proportion to the number of steps, and a risk of degradation in wiring reliability.

In contrast, in the semiconductor device 100, on the same SOI substrate, the back-to-back diode is constituted by the first diode 130 and the second diode 150, and both the diodes are connected to the common ground electrode 175. With this, wire routing is simplified. As a result, improvement in yield, cost reduction, and enhancement in wiring reliability can be achieved.

Note that, the impedance matching circuit of the ultrasonic transducer is a utilization example of the semiconductor device 100, and the semiconductor device 100 can be utilized as various circuits that are formed on the SOI substrate and include the protection circuits.

### Advantages of Semiconductor Device

As described above, the semiconductor device 100 is provided by forming the LV circuit 110, the first diode 130, and the second diode 150 on the single SOI substrate. Channel regions without the BOX layer 202 are formed on the SOI substrate 200, and the first diode 130 and the second diode 150 are formed therein. In this way, the first diode 130 and the second diode 150 to serve as the TR switch can be formed. With this, surge charge is easily discharged.

Further, the element separation region 173 between the first diode 130 and the second diode 150 has a through-trench structure penetrating from the top surface 100a to the bottom surface 100b. With this, “latch-up free,” that is, prevention of latch-up (short-circuiting) can be achieved.

In addition, the first diode 130 and the second diode 150 are each made of the monocrystalline silicon. With this, satisfactory leakage-current characteristics are secured, and a function of the protection circuit is enhanced. Specifically, polysilicon has a mobility of from 1 to 10 cm²/Vs, and the crystalline silicon has a mobility of from approximately 500 to 1,000 cm²/Vs. In depletion regions, the crystalline silicon has a lower resistance of from approximately 1/100 to 1/500.

In particular, a resistance of an “i” layer (layer that is located at a boundary between the N-type semiconductor region and the P-type semiconductor region and has a low dopant density) is problematic. In the “i” layer (P: 1×10⁻¹⁴/cm³) having a thickness of 1 μm and an area of 25² μm², a resistance of polysilicon is 998 Ω), and a resistance of the crystalline silicon is 2 Ω. Thus, in a case where a diode that allows a forward current of 2 A at 200 V to flow therethrough is prepared, a polysilicon diode needs to have an area of 6,242 μm², but the crystalline-silicon diode needs to have an area of 12.5 μm².

Thus, in a case where the diode that is prepared from polysilicon needs to have an area of 80 μm□, the diode that is prepared from the crystalline silicon needs to have an area of only 4 μm□. Generally, in semiconductor devices including the diodes prepared from polysilicon, the diodes need to be formed with use of a plurality of through-trenches. In contrast, the semiconductor device 100 according to this embodiment only needs to have a single diode of 5×5 μm□, and hence an area of implementing the semiconductor device 100 can be reduced.

### Manufacturing Method 1 for Semiconductor Device

A manufacturing method for the semiconductor device 100 is described. As described above, the semiconductor device 100 can be prepared from the SOI substrate 200 (refer to FIG. 3).

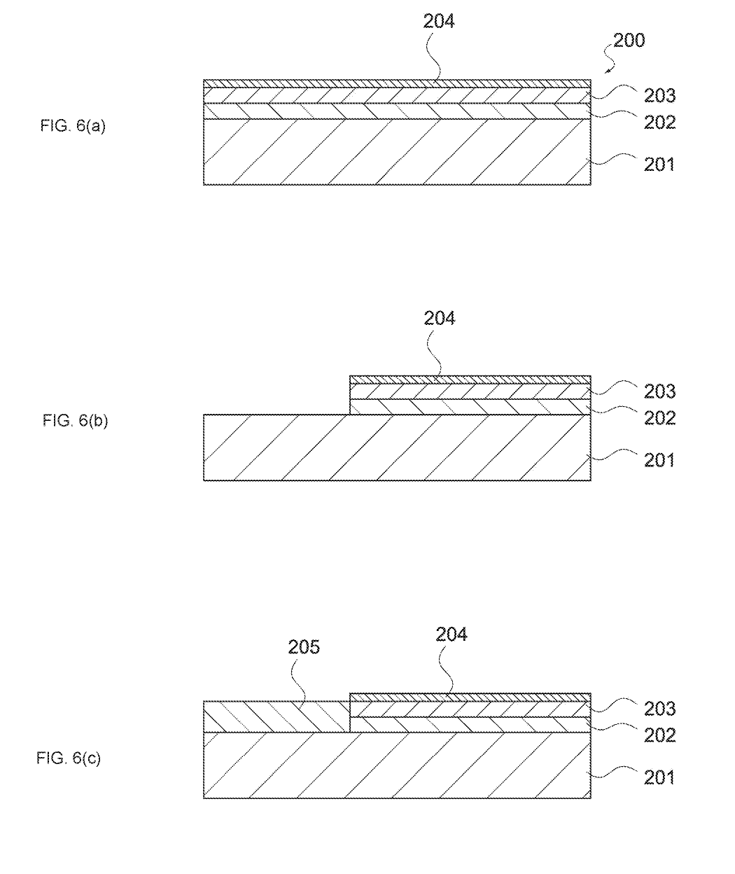

FIG. 6 to FIG. 10 are each a schematic view illustrating the manufacturing method for the semiconductor device 100. As illustrated in (a) of FIG. 6, a sacrificial layer 204 is laminated on the SOI layer 203 of the SOI substrate 200. The sacrificial layer 204 is made of, for example, SiO₂. Then, as illustrated in (b) of FIG. 6, the sacrificial layer 204, the SOI layer 203, and the BOX layer 202 are removed by, for example, etching such that the silicon substrate 201 is exposed.

Next, as illustrated in (c) of FIG. 6, on the silicon substrate 201, crystalline silicon 205 is grown by the epitaxial crystal growth method. By the epitaxial crystal growth method, the silicon substrate 201 and the crystalline silicon 205 have the same crystal orientation.

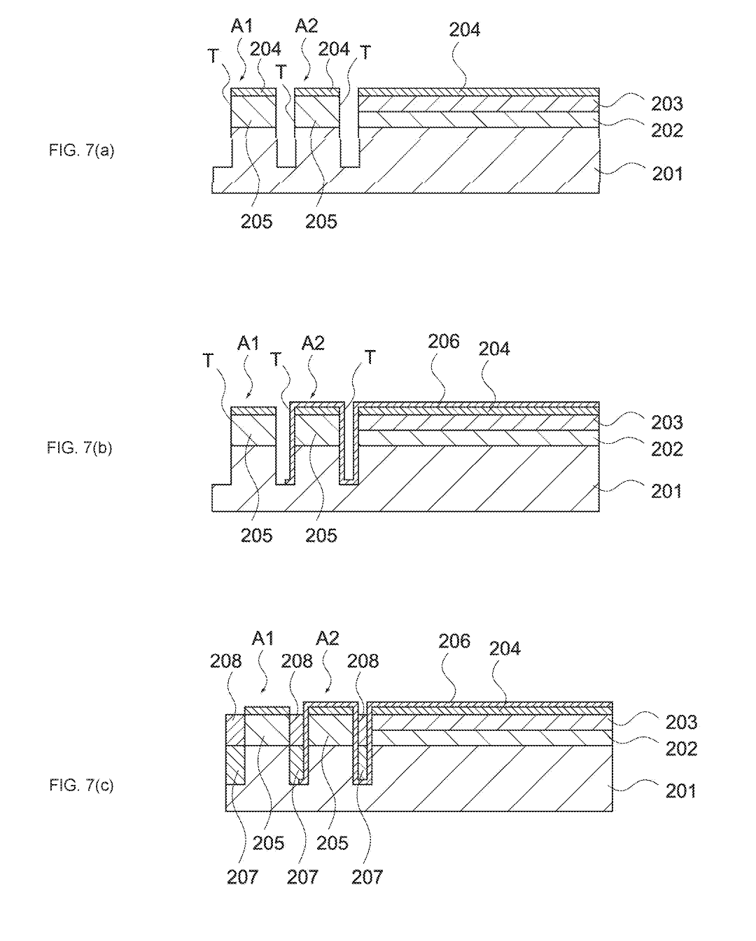

After that, as illustrated in (a) of FIG. 7, the sacrificial layer 204 is laminated on the crystalline silicon 205, and trenches T are formed. The trenches T are formed from the crystalline silicon 205 to the silicon substrate 201, and may each have a depth of approximately several ten μm. By the trenches T, parts of the silicon substrate 201 and the crystalline silicon 205 are separated from each other. With this, a structure A1 and a structure A2 are formed.

Then, as illustrated in (b) of FIG. 7, a diffusion preventing layer 206 is laminated on the sacrificial layer 204 and in the trenches T, and then patterned such that the structure A1 is exposed. The diffusion preventing layer 206 is made of, for example, silicon nitride.

Next, as illustrated in (c) of FIG. 7, PSG (Phosphorus Silicon Glass) 207 and BSG (Boron Silicon Glass) 208 are filled in the trenches T. At the time of filling, HDP (High Density Plasma) can be used. Alternatively, thin films of the BSG and the PSG may be formed by HDP, and then the BSG and the PSG may be laminated on each other by CVD.

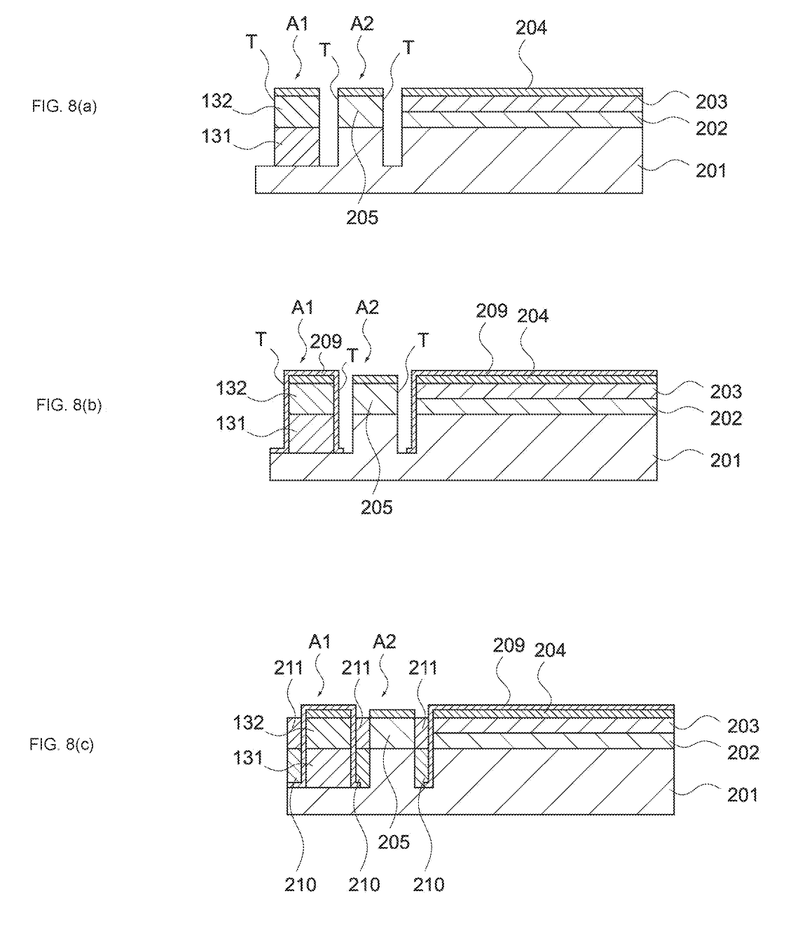

After that, solid-phase diffusion is performed such that, as illustrated in (a) of FIG. 8, the structure Al is doped with the dopants. In the structure A1, phosphorus from the PSG 207 is doped into a region adjacent to the PSG 207. With this, the N-type semiconductor region 131 is formed. In the structure A1, boron from the BSG 208 is doped into a region adjacent to the BSG 208. With this, the P-type semiconductor region 132 is formed. The solid-phase diffusion can be performed by heating.

Then, as illustrated in (b) of FIG. 8, a diffusion preventing layer 209 is laminated on the sacrificial layer 204 and in an element separation trench T, and then patterned such that the structure A2 is exposed. The diffusion preventing layer 209 is made of, for example, silicon nitride.

Next, as illustrated in (c) of FIG. 8, BSG 210 and PSG 211 are filled in the trench T. At the time of filling, as described above, HDP, CVD, and the like may be used.

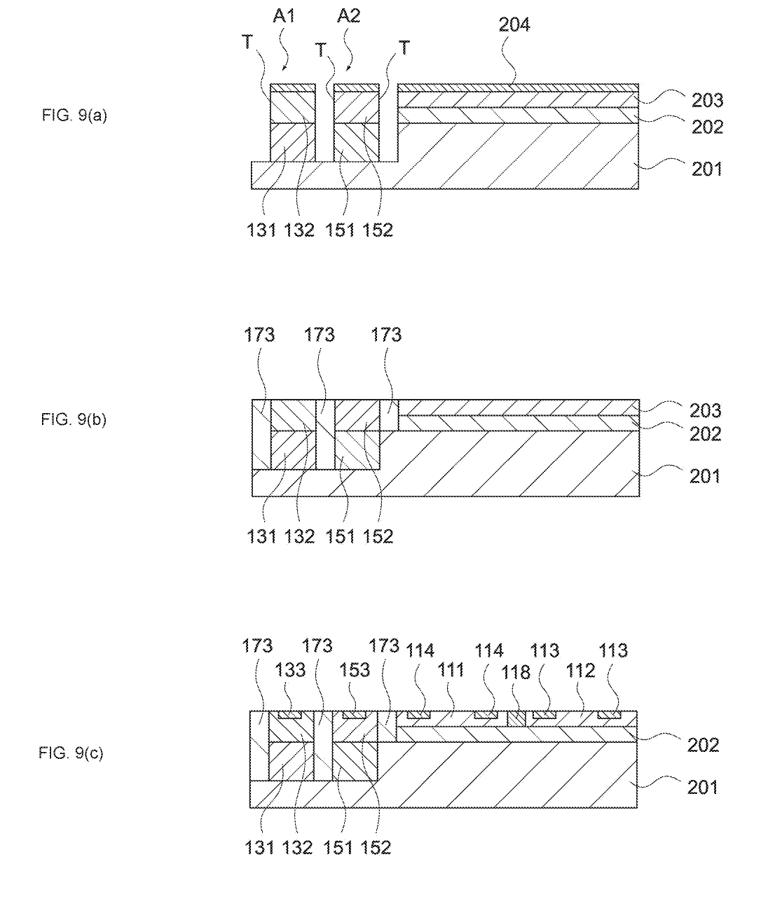

After that, solid-phase diffusion is performed such that, as illustrated in (a) of FIG. 9, the structure A2 is doped with the dopants. In the structure A2, boron from the BSG 210 is doped into a region adjacent to the BSG 210. With this, the P-type semiconductor region 151 is formed. In the structure A2, phosphorus from the PSG 211 is doped into a region adjacent to the PSG 211. With this, the N-type semiconductor region 152 is formed. The solid-phase diffusion can be performed by heating.

Then, as illustrated in (b) of FIG. 9, the element separation region 173 is formed. The element separation region 173 can be formed by filling the material of any one or two or more of silicon oxide, silicon nitride, or polysilicon into the trench T. For example, the element separation region 173 can be formed by forming a film of the insulating materials such as silicon nitride or silicon nitride in the trench T, and then by filling polysilicon into the trench T.

Generally, HDP is used at the time of forming the element separation region 173. However, not only, for example, the PSG and BPSG (Boron Phosphorus Silicon Glass), but also oxide films of, for example, PSG/BPSG, which have high coverage, may be formed by CVD and used in combination therewith. Alternatively, there may be used a combination of HDP and polysilicon, which is widely used in the related art in high-voltage processes for, for example, IGBTs (Insulated Gate Bipolar Transistors).

Next, as illustrated in (c) of FIG. 9, the N-type semiconductor region 111, the P-type semiconductor region 112, the N⁺⁺-type semiconductor regions 113, the P⁺⁺-type semiconductor regions 114, the P⁺⁺-type semiconductor region 133, and the N⁺⁺-type semiconductor region 153 are formed. These can be formed by doping the N-type dopant and the P-type dopant into the SOI layer 203, the P-type semiconductor region 132, and the N-type semiconductor region 152. A method of doping is not particularly limited, and, for example, ion implantation or the solid-phase diffusion may be utilized. Further, as illustrated in (c) of FIG. 9, a part of the SOI layer 203 is oxidized to form the element separation layer 118.

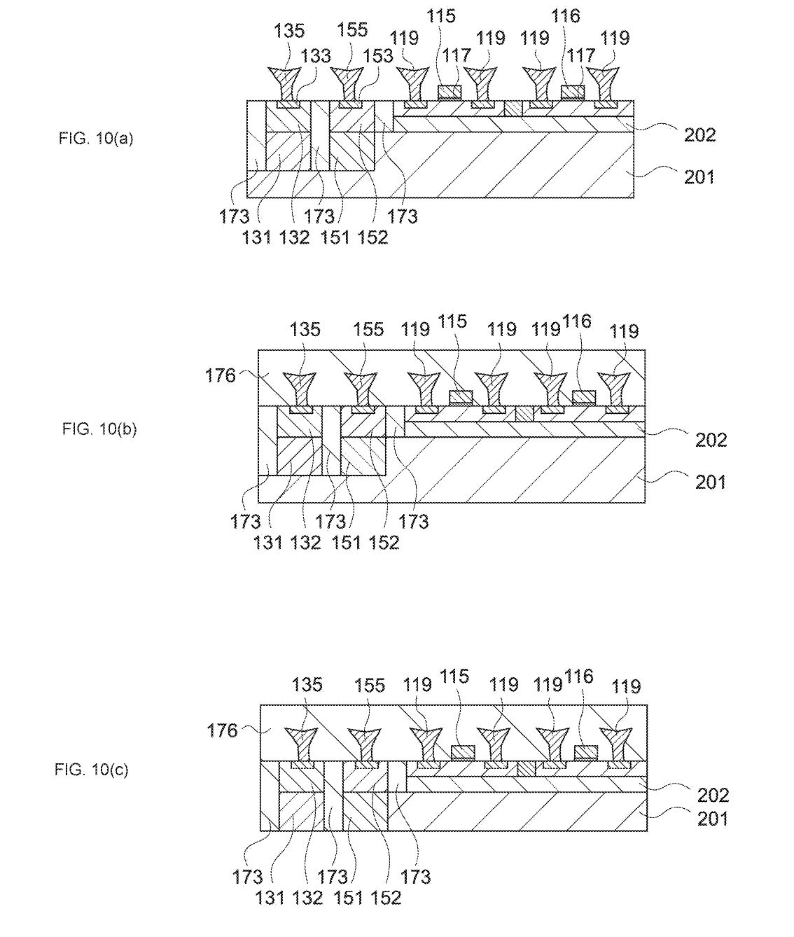

After that, as illustrated in (a) of FIG. 10, the signal wire 135, the signal wire 155, the signal wires 119, the gate insulating films 117, the first gate electrode 115, and the second gate electrode 116 are formed. The gate insulating films 117 can be formed by the oxidization of the SOI layer 203. The signal wire 135, the signal wire 155, the signal wires 119, the first gate electrode 115, and the second gate electrode 116 can be formed by, for example, formation of films of the conductive materials through CVD.

Then, as illustrated in (b) of FIG. 10, the top-surface insulating layer 176 is formed. The top-surface insulating layer 176 can be formed by, for example, CVD. Next, as illustrated in (c) of FIG. 10, a rear surface of the silicon substrate 201 is polished. The polishing is continued until the N-type semiconductor region 131 and the P-type semiconductor region 151 are exposed.

Next, the bottom-surface insulating layer 174, the ground wire 134, the ground wire 154, and the ground electrode 175 are formed (refer to FIG. 1). In order to form the bottom-surface insulating layer 174, a film of TEOS (Tetraethyl orthosilicate) is formed by the plasma-enhanced chemical vapor deposition, and then patterned. TEOS is transformed into SiO₂ by heating. The ground wire 134, the ground wire 154, and the ground electrode 175 can be formed by various metalization processes such as CVD.

The semiconductor device 100 can be prepared in this way. As described hereinabove, a part of the silicon substrate 201 of the SOI substrate 200 serves as the silicon substrate 171 of the semiconductor device 100, and a part of the BOX layer 202 of the SOI substrate 200 serves as the BOX layer 172 of the semiconductor device 100.

### Manufacturing Method 2 for Semiconductor Device

The semiconductor device 100 may be prepared as follows.

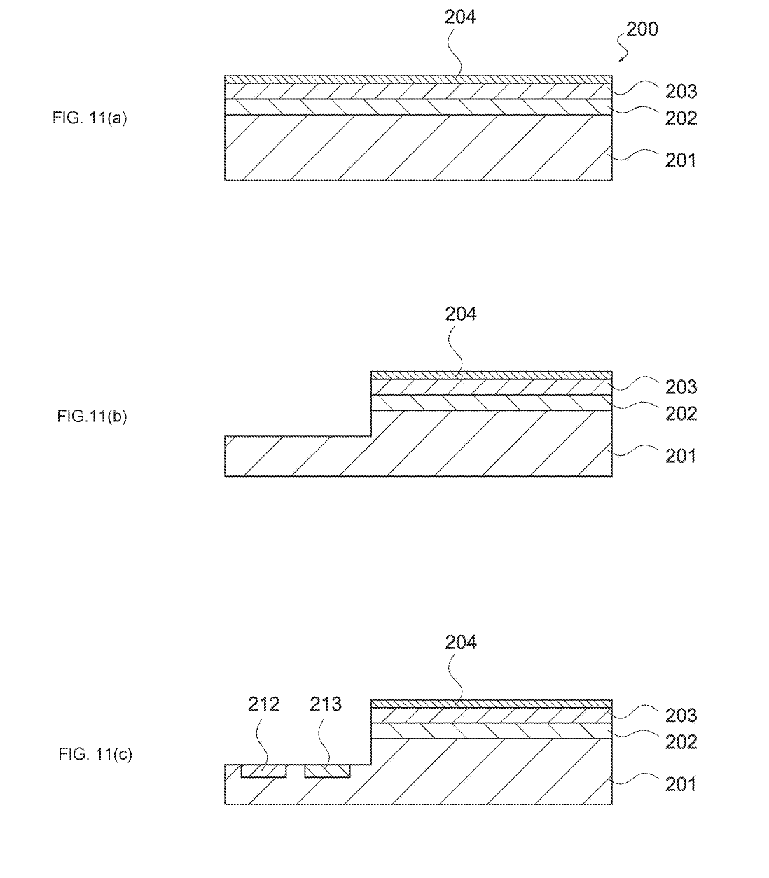

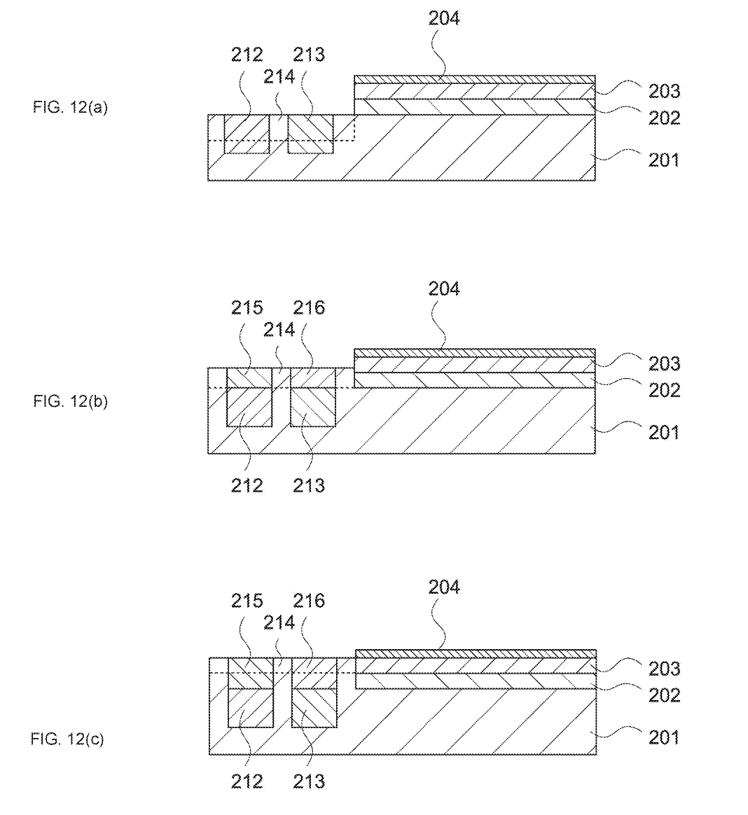

FIG. 11 and FIG. 12 are each a schematic view illustrating another manufacturing method for the semiconductor device 100. As illustrated in (a) of FIG. 11, the sacrificial layer 204 is laminated on the SOI layer 203 of the SOI substrate 200. The sacrificial layer 204 is made of, for example, SiO₂.

Then, as illustrated in (b) of FIG. 11, the sacrificial layer 204, the SOI layer 203, the BOX layer 202, and a part of the silicon substrate 201 are removed by, for example, etching such that the silicon substrate 201 is exposed.

Then, as illustrated in (c) of FIG. 11, an N-type semiconductor region 212 and a P-type semiconductor region 213 are formed. These can be formed by doping the N-type dopant and the P-type dopant into the silicon substrate 201 through the ion implantation or the solid-phase diffusion.

Next, as illustrated in (a) of FIG. 12, crystalline silicon 214 is grown by the epitaxial crystal growth method. At the same time, the dopants are doped by the ion implantation or the solid-phase diffusion so as to form the N-type semiconductor region 212 and the P-type semiconductor region 213. The growth of the crystalline silicon 214 and the doping of the dopants are continued until the N-type semiconductor region 212 and the P-type semiconductor region 213 each have a certain thickness.

After that, as illustrated in (b) of FIG. 12, the crystalline silicon 214 is grown by the epitaxial crystal growth method. At the same time, a P-type semiconductor region 215 and an N-type semiconductor region 216 are formed. The P-type semiconductor region 215 is formed on the N-type semiconductor region 212, and the N-type semiconductor region 216 is formed on the P-type semiconductor region 213. The P-type semiconductor region 215 and the N-type semiconductor region 216 can be formed by doping the dopants through the ion implantation or the solid-phase diffusion. The growth of the crystalline silicon 214 and the doping of the dopants are continued until the P-type semiconductor region 215 and the N-type semiconductor region 216 each have a certain thickness.

(c) of FIG. 12 illustrates a state in which the growth of the crystalline silicon 214 and the doping of the dopants have been completed. The N-type semiconductor region 212 corresponds to the N-type semiconductor region 131 of the first diode 130 (refer to FIG. 2), and the P-type semiconductor region 213 corresponds to the P-type semiconductor region 151 of the second diode 150, respectively. Further, the P-type semiconductor region 215 corresponds to the P-type semiconductor region 132 of the first diode 130, and the N-type semiconductor region 216 corresponds to the N-type semiconductor region 152 of the second diode 150, respectively.

Then, the silicon substrate 201 is oxidized around the first diode 130 and the second diode 150 such that silicon oxide is formed. With this, the element separation region 173 is formed as in (b) of FIG. 9. Subsequently, as in the above-described manufacturing method, the LV circuit 110 and the wires are formed. In this way, the semiconductor device 100 can be prepared.

Note that, the manufacturing methods for the semiconductor device 100 are not limited to those described above as long as the semiconductor device 100 can be manufactured from the SOI substrate 200.

### Modification

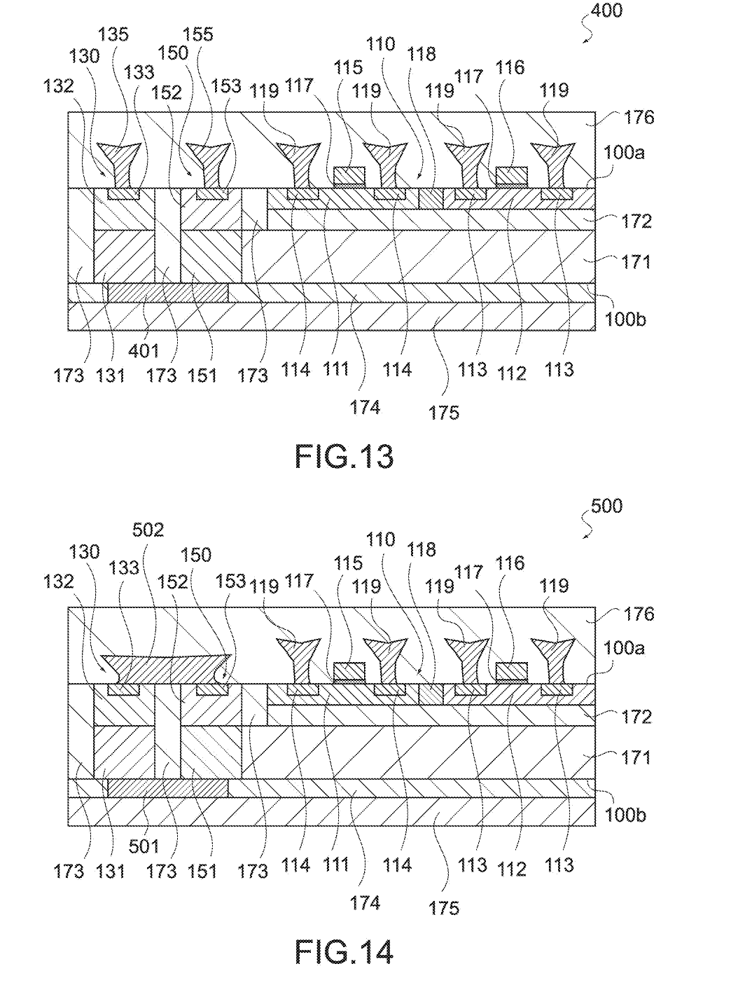

FIG. 13 is a cross-sectional view of a configuration of a semiconductor device 400 according to a first modification of the present technology. As illustrated in FIG. 13, the semiconductor device 400 includes a ground wire 401. Other configuration features of the semiconductor device 400 are the same as those of the semiconductor device 100.

As illustrated in FIG. 13, the ground wire 401 is connected to both the N-type semiconductor region 131 of the first diode 130 and the P-type semiconductor region 151 of the second diode 150, and to the ground electrode 175. Ground potentials of the two diodes constituting the back-to-back diode as shown in FIG. 5 are equal to each other. Thus, the ground wire 401 can be used as a common ground wire for the first diode 130 and the second diode 150. With this, a width of the element separation region 173 is reduced, and hence an area of the semiconductor device 400 can be reduced in accordance therewith.

FIG. 14 is a cross-sectional view of a configuration of a semiconductor device 500 according to a second modification of the present technology. As illustrated in FIG. 14, the semiconductor device 500 includes a ground wire 501 and a signal wire 502. Other configuration features of the semiconductor device 500 are the same as those of the semiconductor device 100.

As illustrated in FIG. 14, the ground wire 501 is connected to both the N-type semiconductor region 131 of the first diode 130 and the P-type semiconductor region 151 of the second diode 150, and to the ground electrode 175. Further, the signal wire 502 is connected to both the P⁺⁺-type semiconductor region 133 of the first diode 130 and the N⁺⁺-type semiconductor region 153 of the second diode 150. The potentials of the signal wires of the two diodes constituting the back-to-back diode as shown in FIG. 5 are equal to each other. Thus, the signal wire 502 can be used as a common signal wire for the first diode 130 and the second diode 150. With this, the width of the element separation region 173 is reduced, and hence an area of the semiconductor device 500 can be reduced in accordance therewith.

### Application Example 1

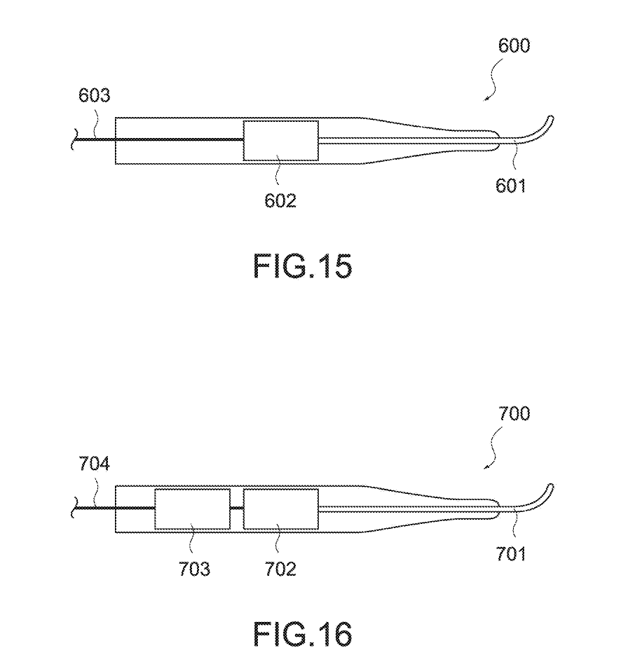

FIG. 15 is a schematic view of a structure of an IVUS (intravascular ultrasound: intravascular ultrasound endoscope) 600 that is capable of utilizing the semiconductor devices 100 according to this embodiment.

As illustrated in FIG. 15, the IVUS 600 includes a catheter 601, an array transducer 602, and a wire 603. The array transducer 602 refers to an array of a plurality of ultrasonic transducer modules. The ultrasonic transducer modules each include the ultrasonic transducer 300 and the impedance matching circuit 301 as shown in FIG. 5. As described above, the impedance matching circuit 301 can be provided in the semiconductor device 100.

When the drive signal is input to the IVUS 600, the drive signal is transmitted to the ultrasonic transducers 300 via the impedance matching circuits 301, and causes the ultrasonic transducers 300 to generate the ultrasonic waves. The generated ultrasonic waves are applied to a vessel wall via the catheter 601 to be inserted into a blood vessel. The reflected waves thereof enter the ultrasonic transducers 300 via the catheter 601, and then are detected. The detection signals are amplified in the impedance matching circuits 301, and then transmitted to a control device of the IVUS 600 via the wire 603.

FIG. 16 is a schematic view of an IVUS 700 having a general structure. As illustrated in FIG. 16, the IVUS 700 includes a catheter 701, an array transducer 702, a signal processing chip 703, and a wire 704. The IVUS 700 operates similar to the IVUS 600, but the impedance matching circuits are installed in the signal processing chip 703.

Generally, in the IVUS, the drive signal is at approximately several ten V, and the detection signals are at approximately several ten μV. The ultrasonic transducers of the IVUS each have a size as significantly small as approximately several ten μm, and hence the signals are difficult to output to an outside of the catheter owing to electrical impedance mismatching. Thus, normally, as illustrated in FIG. 16, the signal processing chip including the impedance matching circuits is provided.

However, the impedance matching circuits are made of silicon, and hence this part is poor in flexibility. As a result, there are difficulties in operating the IVUS. When the semiconductor device 100 according to the present technology is applied, the impedance matching circuits can be integrated with the ultrasonic transducers as illustrated in FIG. 16. With this, the number of inflexible parts is reduced, and hence operability of the IVUS can be increased.

The semiconductor device 100 is applicable not only to the IVUS but also to general integrated circuits using the SOI substrate. Application of the semiconductor device 100 to low-voltage circuits that may be exposed to ESD (electrostatic discharge) or intentionally-generated high-voltage pulses is likely to be especially advantageous.

### Application Example 2

FIG. 20 is a schematic view of a structure of an intraoperative ultrasonic probe 1000 that is capable of utilizing the semiconductor devices 100 according to this embodiment. The intraoperative ultrasonic probe 1000 includes an acoustic lens 1001, an array transducer 1002, and wires 1003. The array transducer 1002 refers to an array of a plurality of ultrasonic transducer modules. The ultrasonic transducer modules each include the ultrasonic transducer 300 and the semiconductor device 100. The semiconductor device 100 constitutes the impedance matching circuit 301 as shown in FIG. 5.

When the drive signal is input to the intraoperative ultrasonic probe 1000, the drive signal is transmitted to the ultrasonic transducers 300 via the impedance matching circuits, and causes the ultrasonic transducers 300 to generate the ultrasonic waves. The generated ultrasonic waves are applied to a diagnostic target through the acoustic lens 1001. The reflected waves thereof enter the ultrasonic transducers 300 through the acoustic lens 1001, and then are detected. The detection signals are amplified in the impedance matching circuits, and then transmitted to a control device of the intraoperative ultrasonic probe 1000 via the wires 1003.

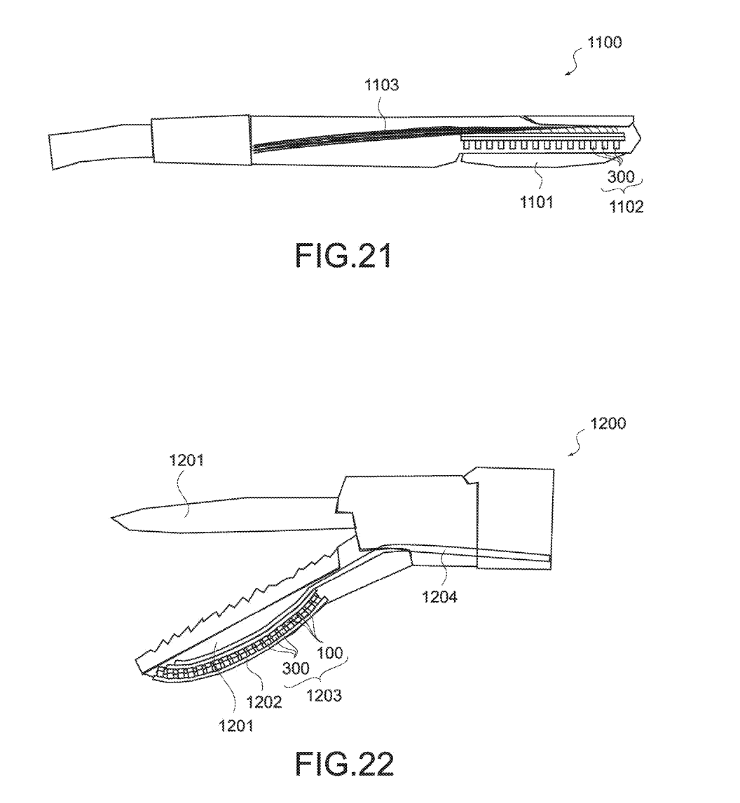

FIG. 21 is a schematic view of an intraoperative ultrasonic probe 1100 having a general structure. As illustrated in FIG. 21, the intraoperative ultrasonic probe 1100 includes an acoustic lens 1101, an array transducer 1102, and wires 1103. The array transducer 1102 refers to an array of a plurality of ultrasonic transducer modules. The ultrasonic transducer modules each include the ultrasonic transducer 300. The intraoperative ultrasonic probe 1100 operates similar to the intraoperative ultrasonic probe 1000, but the array transducer 1102 does not include the semiconductor devices constituting the impedance matching circuits.

The intraoperative ultrasonic wave also transmits the signals at several ten V, and receives the signals at least at several ten μV. The intraoperative ultrasonic probe also uses the array transducer in general, and hence the ultrasonic transducers thereof each have a size as significantly small as approximately several ten μm. In particular, for ease of operation with forceps, further downsizing of intraoperative ultrasonic probes of a drop-in type has been demanded. Thus, the signals have become more difficult to output to an outside of the intraoperative ultrasonic probe owing to the electrical impedance mismatching.

By application of the present technology, an area of the amplifier circuit itself is reduced, and hence a pitch between the ultrasonic transducers can be further reduced. With this, despite downsizing of the array transducer 1002 as illustrated in FIG. 20, degradation of probe performance owing to the impedance mismatching can be restrained. Note that, similar advantages can be expected also when the present technology is applied to the ultrasonic endoscope.

### Application Example 3

FIG. 22 is a schematic view of a structure of a laparoscopic surgical holder 1200 that is capable of utilizing the semiconductor devices 100 according to this embodiment. The laparoscopic surgical holder 1200 includes a holding portion 1201, an acoustic lens 1202, an array transducer 1203, and wires 1204. The holding portion 1201 is configured to be capable of holding an object. The array transducer 1203 refers to an array of a plurality of ultrasonic transducer modules installed in the holding portion 1201. The ultrasonic transducer modules each include the ultrasonic transducer 300 and the semiconductor device 100. The semiconductor device 100 constitutes the impedance matching circuit 301 as shown in FIG. 5.

When the drive signal is input to the laparoscopic surgical holder 1200, the drive signal is transmitted to the ultrasonic transducers 300 via the impedance matching circuits, and causes the ultrasonic transducers 300 to generate the ultrasonic waves. The generated ultrasonic waves are applied to a diagnostic target in contact with the acoustic lens 1202. The reflected waves thereof enter the ultrasonic transducers 300, and then are detected. The detection signals are amplified in the impedance matching circuits, and then transmitted to a control device of the laparoscopic surgical holder 1200 via the wires 1204.

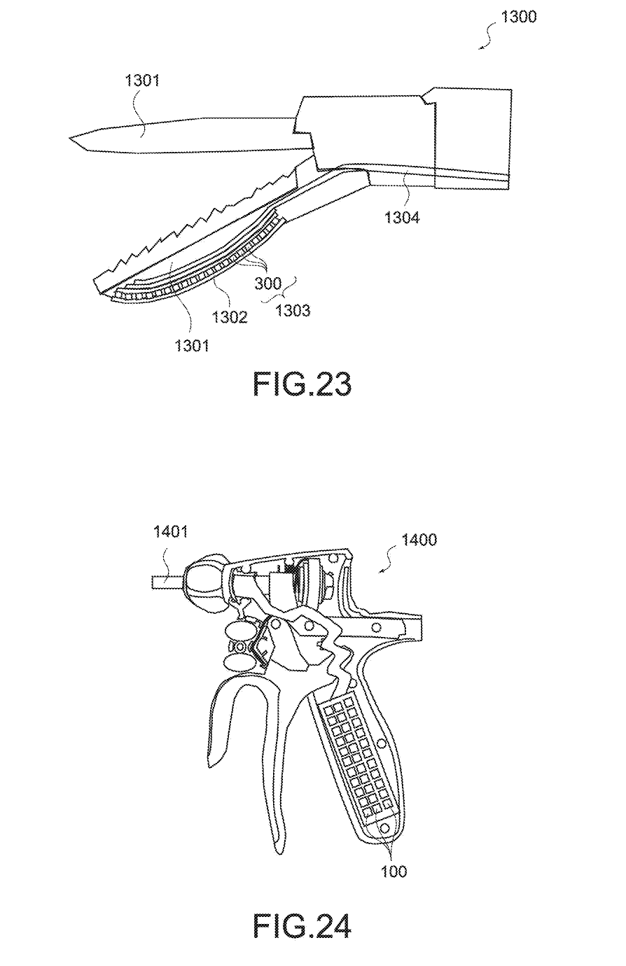

FIG. 23 is a schematic view of a laparoscopic surgical holder 1300 having a general structure. As illustrated in FIG. 23, the laparoscopic surgical holder 1300 includes a holding portion 1301, an acoustic lens 1302, an array transducer 1303, and wires 1304. The array transducer 1303 refers to an array of a plurality of ultrasonic transducer modules. The ultrasonic transducer modules each include the ultrasonic transducer 300. The laparoscopic surgical holder 1300 operates similar to the laparoscopic surgical holder 1200, but the array transducer 1303 does not include the semiconductor devices constituting the impedance matching circuits.

FIG. 24 is a schematic view of a structure of a handle portion of a laparoscopic surgical holder 1400 that is capable of utilizing the semiconductor devices 100 according to this embodiment. The laparoscopic surgical holder 1400 includes a handle portion illustrated in FIG. 24 and a holding portion like the laparoscopic surgical holder 1300 illustrated in FIG. 23. The semiconductor devices 100 constituting the impedance matching circuits of the ultrasonic transducers 300 are installed in the handle portion. The semiconductor devices 100 are connected to the array transducer via a wire 1401.

A surgical instrument including the laparoscopic surgical holder having a distal end at which the ultrasonic probe is incorporated is enabled to perform intraoperative ultrasonic visualization without unnecessary introduction of ports for intraoperative ultrasonic diagnosis. The distal end of the holder is as significantly small as approximately 2×10 mm, and hence characteristic impedance increases. Thus, characteristics are degraded lower than those of existing intraoperative ultrasonic waves.

Thus, application of the present technology to such a holder as illustrated in FIG. 22 and FIG. 24 assists in maintaining performance of the ultrasonic probe. In FIG. 22, the semiconductor devices 100 are installed at the distal end of the holder. It is ideal and advantageous to install the semiconductor devices 100 close to the ultrasonic transducers. However, in a case where the ultrasonic transducers are smaller than the semiconductor devices 100 according to the present technology, the semiconductor devices 100 may be installed in the handle portion of the holder as illustrated in FIG. 24.

### Application Example 4

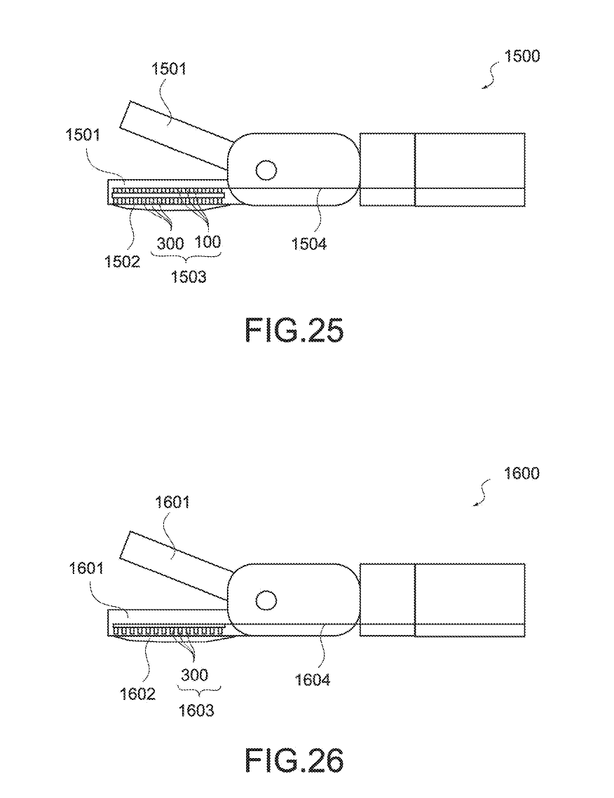

FIG. 25 is a schematic view of a structure of robotic forceps of a laparoscopic-surgery surgical robot 1500 that is capable of utilizing the semiconductor devices 100 according to this embodiment. The laparoscopic-surgery surgical robot 1500 includes a holding portion 1501, an acoustic lens 1502, an array transducer 1503, and a wire 1504. The array transducer 1503 refers to an array of a plurality of ultrasonic transducer modules. The ultrasonic transducer modules each include the ultrasonic transducer 300 and the semiconductor device 100. The semiconductor device 100 constitutes the impedance matching circuit 301 as shown in FIG. 5.

When the drive signal is input to the holding portion 1501, the drive signal is transmitted to the ultrasonic transducers 300 via the impedance matching circuits, and causes the ultrasonic transducers 300 to generate the ultrasonic waves. The generated ultrasonic waves are applied to a diagnostic target in contact with the acoustic lens 1502. The reflected waves thereof enter the ultrasonic transducers 300, and then are detected. The detection signals are amplified in the impedance matching circuits, and then transmitted to a control device of the laparoscopic-surgery surgical robot 1500 via the wire 1504.

FIG. 26 is a schematic view of a structure of robotic forceps of a laparoscopic-surgery surgical robot 1600 having a general structure. As illustrated in FIG. 26, the laparoscopic-surgery surgical robot 1600 includes a holding portion 1601, an acoustic lens 1602, an array transducer 1603, and a wire 1604. The array transducer 1603 refers to an array of a plurality of ultrasonic transducer modules. The ultrasonic transducer modules each include the ultrasonic transducer 300. The laparoscopic-surgery surgical robot 1600 operates similar to the laparoscopic-surgery surgical robot 1500, but the array transducer 1603 does not include the semiconductor devices constituting the impedance matching circuits.

A surgical instrument, that is, the robotic forceps of the laparoscopic-surgery surgical robot, which have a distal end at which the ultrasonic probe is incorporated, is enabled to perform the intraoperative ultrasonic visualization without unnecessary introduction of ports for intraoperative ultrasonic diagnosis. The distal end of the holder is as significantly small as approximately 2×10 mm, and hence characteristic impedance increases. Thus, characteristics are degraded lower than those of the existing intraoperative ultrasonic waves. Thus, application of the present technology to such a holder as illustrated in FIG. 25 assists in maintaining performance of the ultrasonic probe.

As described hereinabove, the semiconductor devices 100 can be utilized in thin and small medical devices such as an intraoperative ultrasonic image pickup device, an ultrasonic catheter, and the ultrasound endoscope. Further, the semiconductor devices 100 can be utilized also in, for example, geodesic ultrasonography and a geodesic ultrasonic sensor, power circuits of millimeter-wave sensors, and control circuits of LEDs (light emitting diodes) for automobiles or projectors, and utilized for, for example, downsizing and implementation of circuits for telecommunications/modems at 48 V/24 V/12 V, downsizing of small-mechatronics control circuits of, for example, a small robot or an endoscope, downsizing of full-digital audio amplifier circuits, and for downsizing of control circuits of HEMSs (home energy management systems).

The utilization of the semiconductor devices 100 enables downsizing of integrated circuits, thereby achieving device downsizing, packaging with amplifier circuits, thereby increasing an SNR (signal-noise ratio), downsizing of solid portions including semiconductor chips, thereby increasing operability of, for example, catheters and endoscopes, and downsizing of the semiconductor chips, thereby increasing yield and theoretical yield. As a result, manufacturing cost can be reduced.

### Second Embodiment

A semiconductor device according to a second embodiment of the present technology is described.

### Configuration of Semiconductor Device

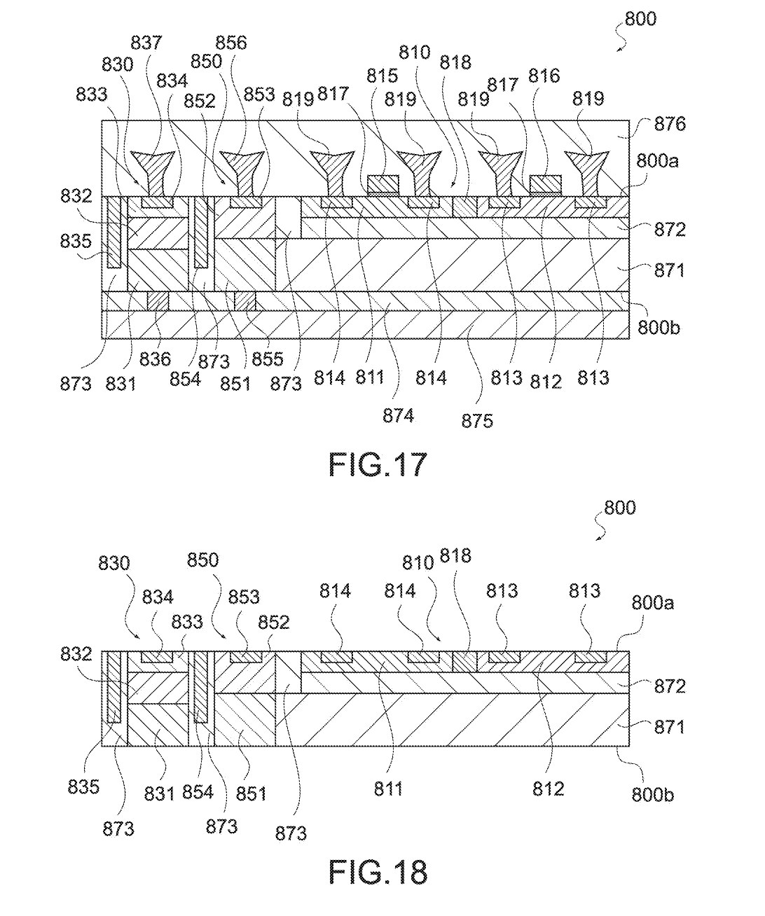

FIG. 17 is a cross-sectional view of a configuration of a semiconductor device 800 according to this embodiment, and FIG. 18 is a cross-sectional view of a part of the configuration of the semiconductor device 800. As illustrated in these drawings, the semiconductor device 800 includes an LV (Low Voltage) circuit 810, a first transistor 830, a second transistor 850, a silicon substrate 871, a BOX layer 872, an element separation region 873, a bottom-surface insulating layer 874, a ground electrode 875, and a top-surface insulating layer 876.

The semiconductor device 800 is an integrated circuit including the LV circuit 810, the first transistor 830, and the second transistor 850. The first transistor 830 and the second transistor 850 serve as protection circuits of this integrated circuit.

As in the first embodiment, the semiconductor device 800 can be prepared from the single SOI substrate 200 (refer to FIG. 3). The silicon substrate 871 of the semiconductor device 800 corresponds to a part of the silicon substrate 201 of the SOI substrate 200, and the BOX layer 872 of the semiconductor device 800 corresponds to a part of the BOX layer 202 of the SOI substrate 200.

In the following, among parts that are formed by processing the SOI substrate 200 of the semiconductor device 800 (structure of FIG. 18), a surface on the LV circuit 810 side is referred to as a top surface 800a, and a surface on a side opposite thereto is referred to as a bottom surface 800b.

The LV circuit 810 includes an N-type semiconductor region 811, a P-type semiconductor region 812, N⁺⁺-type semiconductor regions 813, P⁺⁺-type semiconductor regions 814, a first gate electrode 815, a second gate electrode 816, gate insulating films 817, an element separation layer 818, and signal wires 819.

The N-type semiconductor region 811, which is made of the silicon doped with the N-type dopant, is laminated on the BOX layer 872. A typical N-type dopant is phosphorus. The P-type semiconductor region 812, which is made of the silicon doped with the P-type dopant, is laminated on the BOX layer 872. A typical P-type dopant is boron. The N-type semiconductor region 811 and the P-type semiconductor region 812 are exposed on the top surface 800a, and are separated from each other by the element separation layer 818 made of SiO₂.

The N⁺⁺-type semiconductor regions 813 are made of the silicon doped with the large amount of the N-type dopant, and are formed apart from each other at two positions in the P-type semiconductor region 812. The N⁺⁺-type semiconductor regions 813 are exposed on the top surface 800a, and are connected to the signal wires 819.

The P⁺⁺-type semiconductor regions 814 are made of the silicon doped with the large amount of the P-type dopant, and are formed apart from each other at two positions in the N-type semiconductor region 811. The P⁺⁺-type semiconductor regions 814 are exposed on the top surface 800a, and are connected to the signal wires 819.

The first gate electrode 815 is made of the metals such as aluminum or the conductive materials such as polysilicon, and is formed on the N-type semiconductor region 811 through intermediation of the gate insulating film 817. The second gate electrode 816 is made of the metals such as aluminum or the conductive materials such as polysilicon, and is formed on the P-type semiconductor region 812 through intermediation of the gate insulating film 817.

The N-type semiconductor region 811, the P-type semiconductor region 812, the N⁺⁺-type semiconductor regions 813, and the P⁺⁺-type semiconductor regions 814 are regions formed by implanting the P-type dopant or the N-type dopant into the SOI layer 203 of the SOI substrate 200. The element separation layer 818 is a region formed by oxidizing the SOI layer 203 into SiO₂.

Note that, the configuration of the LV circuit 810 is not limited to the above-described configuration as long as the configuration of the LV circuit 810 can be prepared by processing the SOI layer 203 of the SOI substrate 200.

The first transistor 830 is a vertical transistor including a first P-type semiconductor region 831, an N⁻-type semiconductor region 832, a second P-type semiconductor region 833, a P⁺⁺-type semiconductor region 834, a gate electrode 835, a ground wire 836, and a signal wire 837. The first P-type semiconductor region 831 and the second P-type semiconductor region 833 are each made of the silicon doped with the P-type dopant, and the N⁻-type semiconductor region 832 is made of silicon doped with a small amount of the N-type dopant. The P++-type semiconductor region 834 is made of the silicon doped with the large amount of the P-type dopant.

The first P-type semiconductor region 831, the N⁻-type semiconductor region 832, and the second P-type semiconductor region 833 are laminated in this order. The first P-type semiconductor region 831 is exposed on the bottom surface 800b, and the second P-type semiconductor region 833 is exposed on the top surface 800a. The P⁺⁺-type semiconductor region 834 is formed in the second P-type semiconductor region 833, and is exposed on the top surface 800a.

The first P-type semiconductor region 831, the N⁻-type semiconductor region 832, the second P-type semiconductor region 833, and the P⁺⁺-type semiconductor region 834 are each made of the crystalline silicon, and have the same crystal orientation as the silicon substrate 871. This is because these semiconductor regions are each made of the crystalline silicon that is formed by the implantation of the dopants into parts of the silicon substrate 201, or formed by the epitaxial crystal growth method on the silicon substrate 201.

The gate electrode 835 is embedded in the element separation region 873, and functions as a gate electrode of the first transistor 830. The gate electrode 835 is made of polysilicon. The element separation region 873 may have the structure obtained by forming the film of the insulating materials such as silicon oxide or silicon nitride in a through-hole formed in the SOI substrate 200, and by filling the hole with polysilicon. This polysilicon can be utilized as the gate electrode 835.

On the bottom surface 800b, the ground wire 836 is connected to the first P-type semiconductor region 831. On the top surface 800a, the signal wire 837 is connected to the P⁺⁺-type semiconductor region 834.

The second transistor 850 is a vertical transistor including a P-type semiconductor region 851, an N-type semiconductor region 852, an N⁺⁺-type semiconductor region 853, a gate electrode 854, a ground wire 855, and a signal wire 856. The P-type semiconductor region 851 is made of the silicon doped with the P-type dopant, and the N-type semiconductor region 852 is made of the silicon doped with the N-type dopant. The N⁺⁺-type semiconductor region 853 is made of the silicon doped with the large amount of the N-type dopant.

The P-type semiconductor region 851 and the N-type semiconductor region 852 are laminated on each other. The P-type semiconductor region 851 is exposed on the bottom surface 800b, and the N-type semiconductor region 852 is exposed on the top surface 800a. The N⁺⁺-type semiconductor region 853 is formed in the N-type semiconductor region 852, and is exposed on the top surface 800a.

The P-type semiconductor region 851, the N-type semiconductor region 852, and the N⁺⁺-type semiconductor region 853 are each made of the crystalline silicon, and have the same crystal orientation as the silicon substrate 201. This is because these semiconductor regions are each made of the crystalline silicon that is formed by the implantation of the dopants into the parts of the silicon substrate 201, or formed by the epitaxial crystal growth method on the silicon substrate 201.

The gate electrode 854 is embedded in the element separation region 873, and functions as a gate electrode of the second transistor 850. The gate electrode 854 is made of polysilicon. The element separation region 873 may have the structure obtained by forming the film of the insulating materials such as silicon oxide or silicon nitride in the through-hole formed in the SOI substrate 200, and by filling the hole with polysilicon. This polysilicon can be utilized as the gate electrode 854.

On the bottom surface 800b, the ground wire 855 is connected to the P-type semiconductor region 851. On the top surface 800a, the signal wire 856 is connected to the N⁺⁺-type semiconductor region 853.

The element separation region 873 separates the first transistor 830 and the second transistor 850 from each other. The element separation region 873 penetrates from the top surface 800a to the bottom surface 800b. As in the first embodiment, the element separation region 873 is formed around the first transistor 830 and the second transistor 850 (refer to FIG. 4).

The element separation region 873 is made of the material of any one or two or more of silicon oxide, silicon nitride, or polysilicon. For example, the element separation region 873 may have the structure obtained by forming the film of the insulating materials such as silicon oxide or silicon nitride in the through-hole formed in the SOI substrate 200, and by filling the hole with polysilicon. As described above, the polysilicon can be utilized as the gate electrode 835 and the gate electrode 854.

The bottom-surface insulating layer 874 is arranged on the bottom surface 800b so as to prevent diffusion of moisture or impurity. The bottom-surface insulating layer 874 is made of, for example, p-SiO (silicon oxide formed by plasma-enhanced chemical vapor deposition). The bottom-surface insulating layer 874 is patterned such that the first P-type semiconductor region 831 and the P-type semiconductor region 851 are exposed, and that the bottom-surface insulating layer 174 has opening portions in which the ground wire 836 and the ground wire 855 are formed.

The ground electrode 875 is arranged on the bottom-surface insulating layer 874, and is connected to the ground wire 836 and the ground wire 855. With this, the ground electrode 875 is electrically conducted to the first P-type semiconductor region 831 and the P-type semiconductor region 851. In this way, the ground electrode 875 forms, cooperatively with the ground wire 836 and the ground wire 855, a ground contact structure for the first transistor 830 and the second transistor 850. The ground electrode 875 is made of the conductive materials such as aluminum.

The top-surface insulating layer 876 is arranged on the top surface 800a so as to seal the circuits. The top-surface insulating layer 876 is made of the insulating materials such as SiO₂.

### Utilization Example of Semiconductor Device

FIG. 5 is a schematic diagram of a circuit configuration of an impedance matching circuit 901 of an ultrasonic transducer 900 that is capable of utilizing the semiconductor device 100.

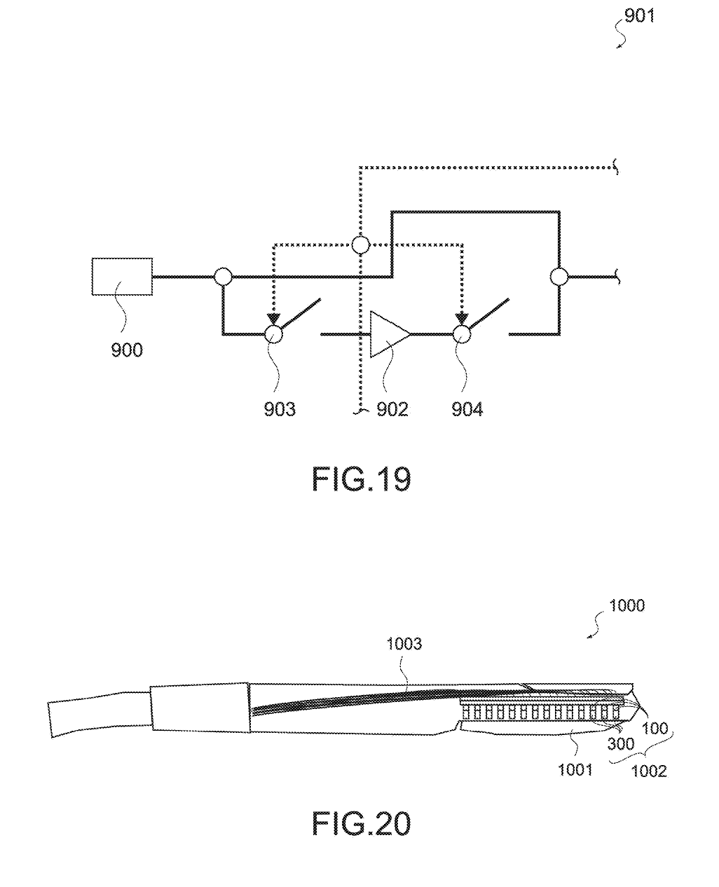

As shown in FIG. 19, the impedance matching circuit 901 includes an amplifier 902, a first TR (transmit-receive) switch 903, and a second TR switch 904.

When a drive signal to the ultrasonic transducer 900 is input with the first TR switch 903 and the second TR switch 904 being turned OFF, the drive signal reaches the ultrasonic transducer 900, and causes the ultrasonic transducer 900 to generate ultrasonic waves. The first TR switch 903 and the second TR switch 904 prevent the drive signal from reaching the amplifier 902.

The first TR switch 903 and the second TR switch 904 are switched ON immediately after the drive signal reaches the ultrasonic transducer 900. When reflected waves of the ultrasonic waves reach the ultrasonic transducer 900, the ultrasonic transducer 900 generates a detection signal. The detection signal reaches the amplifier 902 via the first TR switch 903, is amplified by the amplifier 902, and then output via the second TR switch 904.

In this way, the impedance matching circuit 901 includes the amplifier 902 being an amplifier circuit, the first TR switch 903 and the second TR switch 904 being protection circuits.

In the semiconductor device 800, the LV circuit 810 can be utilized as the amplifier 902, the first transistor 830 can be utilized as the first TR switch 903, and the second transistor 850 can be utilized as the second TR switch 904. With this, the impedance matching circuit 901 can be provided in the single semiconductor device 800.

Note that, the impedance matching circuit of the ultrasonic transducer is a utilization example of the semiconductor device 800, and the semiconductor device 800 can be utilized as various circuits that include the amplifier circuit and the protection circuits.

### Advantages of Semiconductor Device

As described above, the semiconductor device 800 is provided by forming the LV circuit 810, the first transistor 830, and the second transistor 850 on the single SOI substrate. Channel regions without the BOX layer 202 are formed on the SOI substrate 200, and the first transistor 830 and the second transistor 850 are formed therein. In this way, the first transistor 830 and the second transistor 850 to serve as the TR switches can be formed. With this, surge charge is easily discharged.

Further, the element separation region 873 between the first transistor 830 and the second transistor 850 has a through-trench structure penetrating from the top surface 800a to the bottom surface 800b. With this, the “latch-up free” can be achieved.

In addition, the first transistor 830 and the second transistor 850 are each made of the monocrystalline silicon. As in the first embodiment, satisfactory leakage-current characteristics can be secured, the function of the protection circuit can be enhanced, and the area of implementation can be reduced.

### Manufacturing Method for Semiconductor Device

A manufacturing method for the semiconductor device 800 is described. As described above, the semiconductor device 800 can be prepared from the SOI substrate 200 (refer to FIG. 3), and can be manufactured similar to the semiconductor device 100 according to the first embodiment.

The gate electrode 835 and the gate electrode 854 can be prepared by, at the time of preparing the element separation region 873, forming the film of the insulating materials such as silicon nitride or silicon nitride in the trench T (refer to (a) of FIG. 9), filling polysilicon into the trench T, and then routing wires in this polysilicon.

### Application Example

As in the first embodiment, the semiconductor device 800 according to this embodiment can be utilized as the impedance matching circuit of the ultrasonic transducer in the IVUS. The impedance matching circuit can be integrated with the ultrasonic transducer, and hence the operability of the IVUS can be increased. Further, as in the first embodiment, the semiconductor device 800 can be utilized as the impedance matching circuit in various ultrasonic imaging systems including the intraoperative ultrasonic probe, the ultrasound endoscope, the laparoscopic surgical holder, and the laparoscopic-surgery surgical robot.

As in the first embodiment, the semiconductor device 800 is applicable also to general integrated circuits using the SOI substrate. The utilization of the semiconductor device 800 enables downsizing of integrated circuits, thereby achieving device downsizing, packaging with amplifier circuits, thereby increasing an SNR (signal-noise ratio), downsizing of solid portions including semiconductor chips, thereby increasing operability of, for example, catheters and endoscopes, and downsizing of the semiconductor chips, thereby increasing yield and theoretical yield. As a result, manufacturing cost can be reduced.

Note that, the present technology may also provide the following configurations.

(1)

A semiconductor device including an integrated circuit formed on an SOI substrate including
a silicon substrate formed of crystalline silicon,
a BOX (buried oxide) layer laminated on the silicon substrate, and
an SOI (silicon on insulator) layer laminated on the BOX layer, the semiconductor device including:
a protection circuit that configures the integrated circuit and includes a semiconductor region having the same crystal orientation as the silicon substrate; and
an element separation region that penetrates the SOI substrate and separates the protection circuit.

(2)

The semiconductor device according to Item (1), in which the protection circuit is a diode.

(3)

The semiconductor device according to Item (1), in which
the protection circuit is a vertical transistor.

(4)

The semiconductor device according to any one of Items (1) to (3), in which
the element separation region is formed of any one or two or more of silicon oxide, silicon nitride, and polysilicon.

(5)

The semiconductor device according to Item (3), in which
the element separation region includes a gate electrode of the vertical transistor.

(6)

The semiconductor device according to any one of Items (1) to (5), in which
the SOI substrate includes a first surface and a second surface on a side opposite to the first surface,
the protection circuit includes a first semiconductor element and a second semiconductor element,
the first semiconductor element is formed by laminating a first semiconductor region that is on the first surface side and is of a first impurity type, and a second semiconductor region that is on the second surface side and is of a second impurity type, and
the second semiconductor element is formed by laminating a third semiconductor region that is on the first surface side and is of the second impurity type, and a fourth semiconductor region that is on the second surface side and is of the first impurity type.

(7)

The semiconductor device according to Item (6), further including
a ground contact structure that is provided on the first surface of the semiconductor device and is electrically conducted to the first semiconductor region and the third semiconductor region.

(8)

The semiconductor device according to Item (7), in which
the ground contact structure includes a ground wire that is connected to the first semiconductor region and the third semiconductor region and is common to both the first semiconductor region and the third semiconductor region.

(9)

The semiconductor device according to Item (8), in which
the ground contact structure includes a ground electrode that is connected to the ground wire and is common to both the first semiconductor region and the third semiconductor region.

(10)

The semiconductor device according to any one of Items (6) to (9), further including
a signal wire that is connected to the second semiconductor region and the fourth semiconductor region, and is common to both the second semiconductor region and the fourth semiconductor region.

(11)

An ultrasonic image pickup device, including
a semiconductor device including an integrated circuit formed on an SOI substrate including
   a silicon substrate formed of crystalline silicon,
   a BOX layer laminated on the silicon substrate, and
   an SOI layer laminated on the BOX layer, the semiconductor device including
a protection circuit that configures the integrated circuit and includes a semiconductor region having the same crystal orientation as the silicon substrate, and
an element separation region that penetrates the SOI substrate and separates the protection circuit.

(12)

A manufacturing method for a semiconductor device including an integrated circuit formed on an SOI substrate, the manufacturing method including:
preparing the SOI substrate including
   a silicon substrate formed of crystalline silicon,
   a BOX layer laminated on the silicon substrate, and
   an SOI layer laminated on the BOX layer;
forming, by an epitaxial crystal growth method, a protection circuit that configures the integrated circuit and includes a semiconductor region that has the same crystal orientation as the silicon substrate, on the silicon substrate; and
forming an element separation region that penetrates the SOI substrate and separates the protection circuit.

(13)

The manufacturing method for the semiconductor device according to Item (12), in which
in the forming of the protection circuit, a substrate polishing method of polishing the silicon substrate from a surface on a side opposite to a surface on a side where crystal growth of the semiconductor region progresses, to expose the semiconductor region, is used.

(14)

An ultrasonic imaging system, including
an ultrasonic catheter including a semiconductor device including an integrated circuit formed on an SOI substrate including
   a silicon substrate formed of crystalline silicon,
   a BOX layer laminated on the silicon substrate, and
   an SOI layer laminated on the BOX layer, the semiconductor device including
a protection circuit that configures the integrated circuit and includes a semiconductor region having the same crystal orientation as the silicon substrate, and
an element separation region that penetrates the SOI substrate and separates the protection circuit.

(15)

An ultrasonic imaging system, including:
an intraoperative ultrasonic probe or an ultrasound endoscope including a semiconductor device including an integrated circuit formed on an SOI substrate including
   a silicon substrate formed of crystalline silicon,
   a BOX layer laminated on the silicon substrate, and
   an SOI layer laminated on the BOX layer, the semiconductor device including
a protection circuit that configures the integrated circuit and includes a semiconductor region having the same crystal orientation as the silicon substrate, and
an element separation region that penetrates the SOI substrate and separates the protection circuit.

(16)

An ultrasonic imaging system, including
a hand-held instrument that has an ultrasonic imaging function and is used in laparoscopic surgery, the hand-held instrument including a semiconductor device including an integrated circuit formed on an SOI substrate including a silicon substrate formed of crystalline silicon,
   a BOX layer laminated on the silicon substrate, and
   an SOI layer laminated on the BOX layer, the semiconductor device including
a protection circuit that configures the integrated circuit and includes a semiconductor region having the same crystal orientation as the silicon substrate, and
an element separation region that penetrates the SOI substrate and separates the protection circuit.

(17)

An ultrasonic imaging system, including
robotic forceps that have an ultrasonic imaging function and are used in laparoscopic surgery, the robotic forceps including a semiconductor device including an integrated circuit formed on an SOI substrate including
   a silicon substrate formed of crystalline silicon,
   a BOX layer laminated on the silicon substrate, and
   an SOI layer laminated on the BOX layer, the semiconductor device including
a protection circuit that configures the integrated circuit and includes a semiconductor region having the same crystal orientation as the silicon substrate, and
an element separation region that penetrates the SOI substrate and separates the protection circuit.

### REFERENCE SIGNS LIST

100 semiconductor device

110 LV circuit

130 first diode

150 second diode

171 silicon substrate

172 BOX layer

173 element separation region

175 ground electrode

200 SOI substrate

201 silicon substrate

202 BOX layer

203 SOI layer

800 semiconductor device

810 LV circuit

830 first transistor

850 second transistor

871 silicon substrate

872 BOX layer

873 element separation region

875 ground electrode

1. A semiconductor device including an integrated circuit formed on an SOI substrate including
a silicon substrate formed of crystalline silicon,
a BOX (buried oxide) layer laminated on the silicon substrate, and
an SOI (silicon on insulator) layer laminated on the BOX layer, the semiconductor device comprising:
a protection circuit that configures the integrated circuit and includes a semiconductor region having the same crystal orientation as the silicon substrate; and
an element separation region that penetrates the SOI substrate and separates the protection circuit.

2. The semiconductor device according to claim 1, wherein
the protection circuit is a diode.

3. The semiconductor device according to claim 1, wherein
the protection circuit is a vertical transistor.

4. The semiconductor device according to claim 1, wherein
the element separation region is formed of any one or two or more of silicon oxide, silicon nitride, and polysilicon.

5. The semiconductor device according to claim 3, wherein
the element separation region includes a gate electrode of the vertical transistor.

6. The semiconductor device according to claim 1, wherein
the SOI substrate includes a first surface and a second surface on a side opposite to the first surface,
the protection circuit includes a first semiconductor element and a second semiconductor element,
the first semiconductor element is formed by laminating a first semiconductor region that is on the first surface side and is of a first impurity type, and a second semiconductor region that is on the second surface side and is of a second impurity type, and
the second semiconductor element is formed by laminating a third semiconductor region that is on the first surface side and is of the second impurity type, and a fourth semiconductor region that is on the second surface side and is of the first impurity type.

7. The semiconductor device according to claim 6, further comprising
a ground contact structure that is provided on the first surface of the semiconductor device and is electrically conducted to the first semiconductor region and the third semiconductor region.

8. The semiconductor device according to claim 7, wherein
the ground contact structure includes a ground wire that is connected to the first semiconductor region and the third semiconductor region and is common to both the first semiconductor region and the third semiconductor region.

9. The semiconductor device according to claim 8, wherein
the ground contact structure includes a ground electrode that is connected to the ground wire and is common to both the first semiconductor region and the third semiconductor region.

10. The semiconductor device according to claim 6, further comprising
a signal wire that is connected to the second semiconductor region and the fourth semiconductor region and is common to both the second semiconductor region and the fourth semiconductor region.

11. An ultrasonic image pickup device, comprising
a semiconductor device including an integrated circuit formed on an SOI substrate including
a silicon substrate formed of crystalline silicon,
a BOX layer laminated on the silicon substrate, and
an SOI layer laminated on the BOX layer, the semiconductor device including
a protection circuit that configures the integrated circuit and includes a semiconductor region having the same crystal orientation as the silicon substrate, and
an element separation region that penetrates the SOI substrate and separates the protection circuit.

12. A manufacturing method for a semiconductor device including an integrated circuit formed on an SOI substrate, the manufacturing method comprising:
preparing the SOI substrate including
a silicon substrate formed of crystalline silicon,
a BOX layer laminated on the silicon substrate, and
an SOI layer laminated on the BOX layer;
forming, by an epitaxial crystal growth method, a protection circuit that configures the integrated circuit and includes a semiconductor region that has the same crystal orientation as the silicon substrate, on the silicon substrate; and
forming an element separation region that penetrates the SOI substrate and separates the protection circuit.

13. The manufacturing method for a semiconductor device according to claim 12, wherein
in the forming of the protection circuit, a substrate polishing method of polishing the silicon substrate from a surface on a side opposite to a surface on a side where crystal growth of the semiconductor region progresses, to expose the semiconductor region, is used.

14. An ultrasonic imaging system, comprising
an ultrasonic catheter including a semiconductor device including an integrated circuit formed on an SOI substrate including
a silicon substrate formed of crystalline silicon,
a BOX layer laminated on the silicon substrate, and
an SOI layer laminated on the BOX layer, the semiconductor device including
a protection circuit that configures the integrated circuit and includes a semiconductor region having the same crystal orientation as the silicon substrate, and
an element separation region that penetrates the SOI substrate and separates the protection circuit.

15. An ultrasonic imaging system, comprising:
an intraoperative ultrasonic probe or an ultrasound endoscope including a semiconductor device including an integrated circuit formed on an SOI substrate including
a silicon substrate formed of crystalline silicon,
a BOX layer laminated on the silicon substrate, and
an SOI layer laminated on the BOX layer, the semiconductor device including
a protection circuit that configures the integrated circuit and includes a semiconductor region having the same crystal orientation as the silicon substrate, and
an element separation region that penetrates the SOI substrate and separates the protection circuit.

16. An ultrasonic imaging system, comprising
a hand-held instrument that has an ultrasonic imaging function and is used in laparoscopic surgery, the hand-held instrument including a semiconductor device including an integrated circuit formed on an SOI substrate including
a silicon substrate formed of crystalline silicon,
a BOX layer laminated on the silicon substrate, and
an SOI layer laminated on the BOX layer, the semiconductor device including
a protection circuit that configures the integrated circuit and includes a semiconductor region having the same crystal orientation as the silicon substrate, and
an element separation region that penetrates the SOI substrate and separates the protection circuit.

17. An ultrasonic imaging system, comprising
robotic forceps that have an ultrasonic imaging function and are used in laparoscopic surgery, the robotic forceps including a semiconductor device including an integrated circuit formed on an SOI substrate including
a silicon substrate formed of crystalline silicon,
a BOX layer laminated on the silicon substrate, and
an SOI layer laminated on the BOX layer, the semiconductor device including
a protection circuit that configures the integrated circuit and includes a semiconductor region having the same crystal orientation as the silicon substrate, and
an element separation region that penetrates the SOI substrate and separates the protection circuit.
